(19) United States
(12) Patent Application Publication
(10) Pub. No.: US 2019/0030139 A1
(43) Pub. Date: Jan. 31, 2019

(54) USP10 MODULATION OF AMPK AND MTOR

(71) Applicant: MAYO FOUNDATION FOR MEDICAL EDUCATION AND RESEARCH, Rochester, MN (US)

(72) Inventors: Zhenkun LOU, Rochester, MN (US); Min DENG, Rochester, MN (US); Bo QIN, Rochester, MN (US)

(73) Assignee: Mayo Foundation for Medical Education and Research, Rochester, MN (US)

(21) Appl. No.: 16/071,733

(22) PCT Filed: Jan. 20, 2017
(86) PCT No.: PCT/US2017/014286

§ 371 Date: Jul. 20, 2018

Related U.S. Application Data
(60) Provisional application No. 62/281,472, filed on Jan. 21, 2016.

(51) Int. Cl. A61K 38/48

(52) U.S. Cl. CPC A61K 38/4873; C12Y 304/19012

(57) ABSTRACT

Materials and methods for modulating activity of kinases such as AMPK and mTOR via the deubiquitinase, USP10, are provided herein. Also provided are materials and methods for treating clinical conditions mediated at least in part by such kinases, including obesity, diabetes, metabolic syndrome, and some cancers.

### CROSS-REFERENCE TO RELATED APPLICATIONS

This application claims priority from U.S. Provisional Application Ser. No. 62/281,472, filed Jan. 21, 2016.

### TECHNICAL FIELD

This document relates to materials and methods for modulating AMPK and mTOR activity via the deubiquitinase, USP10.

### BACKGROUND

The AMP-activated protein kinase (AMPK) is the master regulator of metabolic homeostasis, serving as a crucial sensor of the cellular response to low energy. AMPK is activated when intracellular ATP concentrations decrease and AMP or ADP concentrations increase in response to energy stresses and pathological stresses (Carling et al., Nature Chem Biol 7:512-518, 2011; Hardie et al., Nature Rev Mol Cell Biol 13:251-262, 2012; Oakhill et al., Trends Endocrinol Metab 23:125-132, 2012). In mammals, AMPK plays critical roles in many metabolic processes, including glucose uptake and fatty acid oxidation in muscle, fatty acid synthesis and gluconeogenesis in the liver, and the regulation of food intake in the hypothalamus (Hardie, supra; Mihaylova and Shaw, Nature Cell Biol 13:1016-1023, 2011; Ruderman et al., J Clin Invest 123:2764-2772, 2013).

AMPK exists as an obligate heterotrimer, composed of the catalytic kinase α subunit and two associated regulatory subunits, β and γ (Carling et al., J Biol Chem 269:11442-11448, 1994; Davies et al., Eur J Biochem 223:351-357, 1994; Michell et al., J Biol Chem 271:28445-28450, 1996; Stapleton et al., J Biol Chem 269:29343-29346, 1994). Upon energy stress, AMP or ADP can directly bind to tandem repeats of crystathionine-β synthase (CBS) domains in the AMPK γ subunit, causing a conformational change that exposes the activation loop in the a subunit, allowing it to be phosphorylated by an upstream kinase and inhibiting dephosphorylation (Davies et al., Febs Lett 377:421-425, 1995; Li et al., Cell Res 25:50-66, 2015; Riek et al., J Biol Chem 283:18331-18343, 2008; Sanders et al., Biochem J 403:139-148, 2007). LKB1 is the major kinase for AMPKα in response to energy stress, and phosphorylates AMPKα at Thr172 (Hawley et al., J Biol 2:28, 2003; Hong et al., Proc Natl Acad Sci USA 100:8839-8843, 2003; Shaw et al., Proc Natl Acad Sci USA 101:3329-3335, 2004; Woods et al., Curr Biol 13:2004-2008, 2003). AMPKα also can be phosphorylated at Thr172 by CaMKKβ, in response to calcium flux (Hawley et al., Cell Metab 2:9-19, 2005; Hurley et al., J Biol Chem 280:29060-29066, 2005; Woods et al., Cell Metab 2:21-33, 2005).

The mammalian target of rapamycin (mTOR, also known as the mechanistic target of rapamycin or FK506-binding protein 12-rapamycin-associated protein 1, FRAP1), is a serine/threonine protein kinase that belongs to the phosphatidylinositol 3-kinase-related kinase protein family. mTOR regulates cell growth, cell proliferation, cell motility, cell survival, protein synthesis, autophagy, and transcription (Hay and Sonenberg, Genes Dev 18:1926-1945, 2004). mTOR integrates input from upstream pathways, including insulin, growth factors (e.g., IGF-1 and IGF-2), and amino acids, and also senses cellular nutrient, oxygen, and energy levels (Tokunaga et al., Biochem Biophys Res Commun 313:443-446, 2004). The mTOR pathway is dysregulated in human diseases such as diabetes, obesity, depression, and certain cancers (Beevers et al., Int J Cancer 119:757-764, 2006; and Xu et al., Biochim Biophys Acta 1846:638-654, 2014). In cancer, for example, over-activation of mTOR signaling can contribute to tumor initiation and development in cancers such as breast, prostate, lung, melanoma, bladder, brain, and renal carcinomas.

### SUMMARY

This document is based, at least in part, on the elucidation of a key molecular mechanism by which AMPK activation is amplified under energy stress. The studies described herein unexpectedly revealed that USP10 functions as a key regulator for AMPK activation, and that USP10-deficiency in mouse liver causes multiple metabolic defects. In particular, ubiquitination on AMPKα blocks AMPKα phosphorylation by LKB1, and USP10 specifically removes ubiquitin from AMPKα to facilitate AMPKα activation via phosphorylation by LKB1. Under energy stress, USP10 activity in turn is enhanced through AMPK-mediated phosphorylation of Ser76 of USP10. Thus, USP10 and AMPK form a key feedforward loop, ensuring amplification of AMPK activation in response to fluctuation of cellular energy status. Disruption of this feedforward loop leads to improper AMPK activation and multiple metabolic defects.

This document also is based, at least in part, on the discoveries that USP10 regulates mTOR ubiquitination, and that USP10 regulates the response of cancer cells to the mTOR inhibitor, everolimus. In particular, as described herein, deubiquitination of mTOR by USP10 reduces mTOR's kinase activity, while increased mTOR ubiquitination in USP10 knockout or knockdown cells is associated with increased mTOR activity.

In a first aspect, this document features a method for increasing the activity of AMP-activated protein kinase (AMPK) in a cell, where the method includes contacting the cell with an agent effective to increase the level or deubiquitinase activity of USP10 in the cell. In some embodiments, the agent can be a USP10 polypeptide. The USP10 polypeptide can include the amino acid sequence set forth in SEQ ID NO:2, or can be a functional variant of the USP10 polypeptide having the sequence set forth in SEQ ID NO:2 (e.g., a functional variant having an amino acid sequence that is at least 95% identical to the sequence set forth in SEQ ID NO:2), or a biologically active fragment of the USP10 polypeptide having the sequence set forth in SEQ ID NO:2 (e.g., a biologically active fragment containing amino acid residues 520 to 793 of SEQ ID NO:2). In some embodiments, the agent can be a nucleic acid encoding a USP10 polypeptide. The nucleic acid can include the sequence set forth in nucleotides 143-2536 of SEQ ID NO:1. The nucleic acid can encode a functional variant of a USP10 polypeptide having the sequence set forth in SEQ ID NO:2 (e.g., a functional variant having an amino acid sequence that is at least 95% identical to the sequence set forth in SEQ ID NO:2), or the nucleic acid can encode a biologically active fragment of a USP10 polypeptide having the sequence set forth in SEQ ID NO:2 (e.g., a biologically active fragment comprises amino acid residues 520 to 793 of SEQ ID NO:2). The nucleic acid can be in a viral vector. The cell can be in a subject identified as having a clinical condition selected from the group consisting of obesity, type 2 diabetes, insulin resistance, or metabolic syndrome. For example, the cell can be in a subject identified as having cancer, where the cancer is selected from the group consisting of renal cancers, pancreatic cancers, breast cancers, prostate cancers, lung cancers, melanoma, bladder cancers, and brain cancers. The subject can be a human.

In another aspect, this document features a method for reducing the activity of mammalian target of rapamycin (mTOR) in a cell, where the method includes contacting the cell with an agent effective to increase the level or deubiquitinase activity of USP10 in the cell. In some embodiments, the agent can be a USP10 polypeptide. The USP10 polypeptide can include the amino acid sequence set forth in SEQ ID NO:2, or can be a functional variant of the USP10 polypeptide having the sequence set forth in SEQ ID NO:2 (e.g., a functional variant having an amino acid sequence that is at least 95% identical to the sequence set forth in SEQ ID NO:2), or a biologically active fragment of the USP10 polypeptide having the sequence set forth in SEQ ID NO:2 (e.g., a biologically active fragment containing amino acid residues 520 to 793 of SEQ ID NO:2). In some embodiments, the agent can be a nucleic acid encoding a USP10 polypeptide. The nucleic acid can include the sequence set forth in nucleotides 143-2536 of SEQ ID NO:1. The nucleic acid can encode a functional variant of a USP10 polypeptide having the sequence set forth in SEQ ID NO:2 (e.g., a functional variant having an amino acid sequence that is at least 95% identical to the sequence set forth in SEQ ID NO:2), or the nucleic acid can encode a biologically active fragment of a USP10 polypeptide having the sequence set forth in SEQ ID NO:2 (e.g., a biologically active fragment comprises amino acid residues 520 to 793 of SEQ ID NO:2). The nucleic acid can be in a viral vector. The cell can be in a subject identified as having a clinical condition selected from the group consisting of obesity, type 2 diabetes, insulin resistance, or metabolic syndrome. For example, the cell can be in a subject identified as having cancer, where the cancer is selected from the group consisting of renal cancers, pancreatic cancers, breast cancers, prostate cancers, lung cancers, melanoma, bladder cancers, and brain cancers. The subject can be a human.

In another aspect, this document features a method for reducing cancer cell proliferation in a mammal having cancer cells, where the method includes administering a composition to the mammal under conditions wherein the composition modulates USP10 polypeptide expression or activity within the cancer cells, thereby reducing cancer cell proliferation. The composition can include a USP10 polypeptide containing the amino acid sequence set forth in SEQ ID NO:2, or a functional variant or biologically active fragment thereof. The composition can include a nucleic acid encoding a USP10 polypeptide, where the USP10 polypeptide includes the amino acid sequence set forth in SEQ ID NO:2, or a functional variant or biologically active fragment thereof.

In still another aspect, this document features a method for treating insulin resistance, type 2 diabetes, or metabolic syndrome in a subject in need thereof, where the method includes administering a composition to the mammal under conditions wherein the composition modulates USP10 polypeptide expression or activity within the cells of the subject, thereby reducing a symptom of the insulin resistance, type 2 diabetes, or metabolic syndrome. The composition can include a USP10 polypeptide containing the amino acid sequence set forth in SEQ ID NO:2, or a functional variant or biologically active fragment thereof. The composition can include a nucleic acid encoding a USP10 polypeptide, where the USP10 polypeptide contains the amino acid sequence set forth in SEQ ID NO:2, or a functional variant or biologically active fragment thereof.

This document also features a method for increasing the activity of AMPK in a cell, where the method includes contacting the cell with an agent effective to increase, in the cell, the level of an AMPK polypeptide that lacks one or more ubiquitination sites. In some embodiments, the agent can be an AMPK polypeptide that lacks one or more ubiquitination sites. In some embodiments, the agent can be a nucleic acid encoding an AMPK polypeptide that lacks one or more ubiquitination sites. The AMPK polypeptide can be an AMPKα1 polypeptide having a substitution of one or more of the lysine at position 71, the lysine at position 285, the lysine at position 396, and the lysine at position 485. In some embodiments, an arginine can be substituted for one or more of the lysine at position 71, the lysine at position 285, the lysine at position 396, and the lysine at position 485. For example, an arginine can be substituted for the lysine at position 71, or an arginine can be substituted for each of the lysine at position 71, the lysine at position 285, the lysine at position 396, and the lysine at position 485. In some embodiments, the AMPK polypeptide can be an AMPKα2 polypeptide having a substitution of one or more of the lysine at position 60, the lysine at position 379, the lysine at position 391, and the lysine at position 470. For example, an arginine can be substituted for one or more of the lysine at position 60, the lysine at position 379, the lysine at position 391, and the lysine at position 470. In some embodiments, an arginine can be substituted for each of the lysine at position 60, the lysine at position 379, the lysine at position 391, and the lysine at position 470.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1I demonstrate that USP10 regulates AMPK activation and cellular metabolism. FIG. 1A is a picture of an immunoblot showing the effect of USP10 knockdown on AMPK activation. HCT116 cells were infected with lentivirus encoding the indicated shRNAs. Cell lysates were immunoblotted as indicated. FIGS. 1B and 1C are a picture (FIG. 1B) and a graph (FIG. 1C) showing Oil-Red-O staining of HCT116 cells with or without shRNA knockdown of USP10. Control or USP10 knockdown HCT116 cells were incubated in medium supplemented with 200 μM sodium oleate overnight (20 hours) and fixed with formalin. The intracellular lipid droplet abundance was assessed by Oil-Red-O staining. The results represent the means (±SEM) of three independent experiments. **, p<0.01. FIG. 1D is a graph plotting lactate concentration in HCT116 cells. Lactate production was measured after incubation of control and USP10 knockdown HCT116 cells in medium for 48 hours. The results represent the means (±SEM) of three independent experiments. **, p<0.01. FIG. 1E is a graph plotting relative expression of aldoa, ldha, and pdkl mRNA in control or USP10 knockdown HCT116 cells, as determined by qPCR. Transcript levels were determined relative to actin mRNA levels, and normalized relative to control cells. The results represent the means (±SEM) of three independent experiments. **, p<0.01. FIG. 1F is a picture of an immunoblot showing the effect of USP10 activity or USP10 knockdown on AMPK activation. Cells were transfected with indicated USP10 constructs and placed in medium without glucose for 4 hours. Cell lysates were immunoblotted with the indicated antibodies. FIG. 1G is a graph plotting relative Oil-Red-O staining in cells transfected with the indicated USP10 constructs and subjected or not subjected to shRNA knockdown. Cells as in FIG. 1F were incubated in medium supplemented with 200 μM sodium oleate overnight (20 hours) and placed in medium without glucose for 4 hours. The intracellular lipid droplet abundance was assessed by Oil-Red-O staining. The results represent the means (±SEM) of three independent experiments. **, p<0.01. FIG. 1H is a graph plotting the effect of USP10 deubiquitinase activity on glycolytic gene expression. Cells as in FIG. 1F were placed in medium without glucose for 4 hours. The relative expression of aldoa, ldha, and pdkl mRNA in the indicated cells was determined by qPCR. Transcript levels were determined relative to actin mRNA levels, and normalized relative to control cells. The results represent the means (±SEM) of three independent experiments. **, p<0.01. FIG. 1I is a picture of an immunoblot showing the effect of Spautin-1 treatment on AMPK activation. HCT116 cells were treated with indicated concentration of Spautin-1 and cell lysates were immunoblotted as indicated.

FIGS. 2A-2H demonstrate that USP10 regulates cellular metabolism through AMPK but not through p53. FIG. 2A is a picture of an immunoblot showing the effect of USP10 knockdown on AMPK activation in the absence of p53. HCT116 p53−/− cells were infected with lentivirus encoding the indicated shRNAs. Cell lysates were immunoblotted as indicated. FIG. 2B is a graph plotting relative Oil-Red-O staining in p53−/− cells with and without shRNA knockdown of USP10. Control and USP10 knockdown HCT116 p53−/− cells were incubated in medium supplemented with 200 μM sodium oleate overnight (20 hours) and fixed with formalin. Intracellular lipid droplet abundance was assessed by Oil-Red-O staining. FIG. 2C is a graph plotting lactate production in p53−/− cells with and without shRNA knockdown of USP10. Lactate production was measured after incubation of control and USP10 knockdown HCT116 p53−/− cells in medium for 48 hours. The results represent the means (±SEM) of three independent experiments. **, p<0.01. FIG. 2D is a graph plotting relative mRNA levels for glycolytic genes in p53−/− cells with and without knockdown of USP10. Relative expression of aldoa, ldha, and pdkl mRNA in control or USP10 knockdown HCT116 p53−/− cells was determined by qPCR. Transcript levels were determined relative to actin mRNA levels, and normalized relative to control cells. The results represent the means (±SEM) of three independent experiments. **, p<0.01. FIG. 2E is a picture of an immunoblot of lysates from cells infected with lentivirus encoding shRNAs targeted to USP10, AMPK, or both. Cell lysates were incubated with the indicated antibodies. FIG. 2F is a graph plotting relative Oil-Red-O staining in cells that were infected with lentivirus encoding the indicated shRNAs, incubated in medium supplemented with 200 μM sodium oleate overnight (20 hours), and fixed with formalin. Intracellular lipid droplet abundance was assessed by Oil-Red-O staining. The results represent the means (±SEM) of three independent experiments. **, p<0.01; n.s, not significant. FIG. 2G is a graph plotting lactate production by cells treated with the indicated shRNAs and incubated in medium for 48 hours. The results represent the means (±SEM) of three independent experiments. **, p<0.01; n.s, not significant. FIG. 2H is a graph plotting relative expression of mRNA for the glycolytic genes aldoa, ldha, and pdkl in the indicated cells, as determined by qPCR. Transcript levels were determined relative to actin mRNA levels, and normalized relative to control cells. The results represent the means (±SEM) of three independent experiments. **, p<0.01; n.s, not significant.

FIGS. 3A-3H demonstrate that USP10 interacts with AMPKα and deubiquitinates AMPKα. FIG. 3A is a picture of a blot demonstrating that USP10 directly interacts with AMPKα in vitro. Purified His6-USP10 was incubated with the indicated GST tagged AMPK subunits coupled to GSH-Sepharose. Proteins retained on Sepharose were then blotted with the indicated antibodies. FIGS. 3B-3D are immunoblots showing the interaction of USP10 with AMPKα in cells. HCT116 cell lysates were subjected to immunoprecipitation (IP) with control IgG, anti-USP10 (FIG. 3B), anti-AMPKα1 (FIG. 3C), anti-AMPKα2 (FIG. 3D) antibodies. The immunoprecipitates were then blotted with the indicated antibodies. FIG. 3E is an immunoblot (upper panel) demonstrating the interaction of AMPKα with various portions of USP10 (lower panel). 293T cells transfected with the indicated constructs were lysed, and lysates were incubated with anti-FLAG beads. Proteins retained on beads were blotted with the indicated antibodies. FIG. 3F is a picture of an immunoblot demonstrating that USP10 knockdown increases AMPKα ubiquitination. HCT116 cells infected with lentivirus encoding the indicated shRNAs were transfected with the indicated constructs. Cell lysates and Ni-NTA pulldowns were immunoblotted as indicated. -Glu, glucose starvation. FIG. 3G is a picture of an immunoblot showing that USP10 deubiquitinase activity is required for AMPK deubiquitination. The indicated constructs were transfected into USP10 knockdown cell lines, and cells were placed in medium without glucose for 4 hours. Cell lysates were incubated with anti-HA beads and immunoblotted with the indicated antibodies. -Glu, glucose starvation. FIG. 3H is a picture of an immunoblot showing deubiquitination of AMPKα2 in vitro by USP10. Ubiquitinated AMPKα2 was incubated with purified USP10 or USP10CA in vitro, and then blotted with indicated antibodies.

FIGS. 4A-4C demonstrate that USP10 interacts with AMPKα and deubiquitinates AMPKα. FIG. 4A is a picture of an immunoblot showing the effect of USP10 on AMPKα1 ubiquitination. HCT116 cells infected with lentivirus encoding the indicated shRNAs were transfected with the indicated constructs. Cell lysates and Ni-NTA pulldowns were immunoblotted as indicated. FIG. 4B is a picture of an immunoblot showing the effect of USP10 on AMPKα1 ubiquitination. The indicated constructs were transfected into USP10 knockdown cell lines. Cells were placed in medium without glucose for 4 hours. Cell lysates were incubated with anti-HA beads and immunoblotted with the indicated antibodies. FIG. 4C is a picture of an immunoblot showing deubiquitination of AMPKα1 in vitro by USP10. Ubiquitinated AMPKα1 was incubated with purified USP10 or USP10CA in vitro, and then blotted with indicated antibodies.

FIGS. 5A-5K demonstrate that ubiquitination of AMPKα inhibits its activation. FIG. 5A is a picture of an immunoblot showing the effect of ubiquitin chain type on AMPKα2. AMPKα2 expression vectors and the indicated HA-tagged ubiquitin were transfected into USP10 knockdown cells. Cell lysates were boiled and immunoprecipitated with anti-HA beads and immunoblotted as indicated. FIG. 5B is a picture of an immunoblot showing that knockdown of USP10 increases AMPKα2 K63 ubiquitination. HCT116 cells infected with lentivirus encoding the indicated shRNAs were transfected with the indicated constructs. Cell lysates were boiled and immunoprecipitated with anti-FLAG beads and immunoblotted as indicated. FIG. 5C shows AMPKα sequences containing candidate ubiquitin sites. FIG. 5D is a picture of an immunoblot indicating the ubiquitination sites on AMPKα2. AMPKα2 expression vectors and HA-Ub K63 plasmid were transfected into USP10 knockdown cells. Cell lysates were boiled and immunoprecipitated with FLAG beads and immunoblotted as indicated. FIG. 5E is a picture of immunoblots demonstrating regulation of AMPKα2 ubiquitination by USP10. AMPKα2 expression vectors were transfected into control and USP10 knockdown cells. Cell lysates were boiled and immunoprecipitated with FLAG beads and immunoblotted as indicated. FIGS. 5F and 5G are immunoblots demonstrating that ubiquitination of AMPKα affects LKB1 binding (FIG. 5G) and T172 phosphorylation (FIG. 5F). AMPKα2 expression vectors were transfected into HCT116 cells. Lysates were immunoprecipitated with FLAG beads and immunoblotted as indicated. FIG. 5H is a picture of immunoblots demonstrating that USP10 regulates AMPK activation through deubiquitination of AMPKα. AMPKα2 expression vectors were transfected into control or USP10 knockdown cells. Cell lysates were immunoprecipitated with FLAG beads and immunoblotted as indicated. FIG. 5I is a picture of immunoblots showing that ubiquitination of AMPKα inhibits AMPK activation. Wild-type (WT) or AMPK α1/α2 double knockout (DKO) mouse embryonic fibroblasts (MEFs) were infected with retrovirus expression AMPKα or the 4KR mutant, and placed in media containing vehicle or 2 mM AICAR for 1 hour. Cell lysates were immunoblotted as indicated. FIG. 5J is a graph plotting relative Oil-Red-O staining for AMPK α1/α2 double knockout (DKO) MEFs infected with retrovirus expression AMPKα or the 4KR mutant. Cells were incubated in medium supplemented with 200 μM sodium oleate overnight (20 hours) and placed in medium without glucose for 4 hours. The intracellular lipid droplet abundance was assessed by Oil-Red-O staining. The results represent the means (±SEM) of three independent experiments. **, p<0.01. FIG. 5K is a graph plotting relative mRNA levels for glycolytic genes in AMPK α1/α2 double knockout (DKO) MEFs infected with retrovirus expressing AMPKα or the KR mutant. Cells were placed in medium without glucose for 4 hours. Relative expression of aldoa, ldha, and pdkl mRNA in the indicated cells was determined by qPCR. Transcript levels were determined relative to actin mRNA levels, and normalized relative to control cells. The results represent the means (±SEM) of three independent experiments. **, p<0.01.

FIGS. 6A-6G demonstrate that ubiquitination of AMPKα inhibits its activation. FIG. 6A is a picture of an immunoblot showing the effect of ubiquitin chain type on AMPKα1. AMPKα1 expression vectors and indicated HA tag ubiquitin were transfected into USP10 knockdown cells. Cell lysates were boiled and immunoprecipitated with anti-HA beads, and immunoblotted as indicated. FIG. 6B is a picture of immunoblots showing the effect of USP10 knockdown on AMPKα1 K63 ubiquitination. HCT116 cells infected with lentivirus encoding the indicated shRNAs were transfected with the indicated constructs. Cell lysates were boiled and immunoprecipitated with anti-FLAG beads and immunoblotted as indicated. FIG. 6C is a picture of immunoblots indicating the ubiquitination sites on AMPKα1. AMPKα1 expression vectors and HA-Ub K63 plasmid were transfected into USP10 knockdown cells. Cell lysates were boiled and immunoprecipitated with FLAG beads and immunoblotted as indicated. FIG. 6D is a picture of immunoblots showing the effect of AMPKα1 expression vectors transfected into control or USP10 knockdown cells. Cell lysates were boiled and immunoprecipitated with FLAG beads and immunoblotted as indicated. FIG. 6E is a picture of immunoblots showing the effect of WT or 4KR AMPKα1 expression on AMPK phosphorylation in HCT116 cells. Lysates were immunoprecipitated with FLAG beads and immunoblotted as indicated. FIG. 6F is a picture of immunoblots showing the effect of WT or 4KR AMPKα1 expression on LKB1 in HCT116 cells. Lysates were immunoprecipitated with FLAG beads and immunoblotted as indicated. FIG. 6G is a picture of immunoblots showing the effect of WT or 4KR AMPKα1 expression on protein interactions in control or USP10 knockdown cells. Cell lysates were immunoprecipitated with FLAG beads and immunoblotted as indicated.

FIGS. 7A-7D demonstrate that glucose starvation increases USP10 activity. FIGS. 7A and 7B are immunoblots showing the effect of glucose starvation on ubiquitination of AMPKα1 (FIG. 7A) and AMPKα2 (FIG. 7B). AMPKα and Ub expression vectors were transfected into HCT116 cells, and cells were placed in medium with or without glucose for 4 hours. Cell lysates were boiled and immunoprecipitated with FLAG beads and immunoblotted as indicated. FIG. 7C is a graph plotting the USP10 deubiquitinase activity. Cells transfected with FLAG-USP10 expression vector were placed in medium with or without glucose for 4 hours. Cell lysates were incubated with FLAG beads and then eluted with FLAG peptide. The activities of eluted proteins were assayed using ubiquitin-7-amido-4-methylcoumarin (Ub-AMC) as a substrate. FIG. 7D is a picture of immunoblots demonstrating USP10 deubiquitinating activity in cells cultured in medium with or without glucose for 4 hours. Cells were lysed in lysis buffer and total protein was labeled with Ub-VS probe, then subjected to immunoblotting with the indicated antibodies.

FIGS. 8A-8G demonstrate that AMPK phosphorylates USP10 at Ser76. FIG. 8A is a Clustal alignment of conserved sites in USP10 from various species that match the optimal AMPK substrate motif. FIG. 8B is a picture of immunoblots demonstrating that USP10 is phosphorylated under energy stress. USP10 expression vectors were transfected into HCT116 cells. Cells were placed in medium with or without glucose for 4 hours. FLAG-USP10 was immunoprecipitated and treated with or without λ-PPase, then blotted with the indicated antibodies. -Glu, glucose starvation. FIG. 8C is a picture of a gel showing phosphorylation of USP10 by radiolabeled AMPK in vitro. Bacterially expressed GST and USP10 were incubated with purified AMPK in the presence of [γ-³²P]ATP with or without AMP. Proteins were resolved by SDS-PAGE; proteins were visualized by Coomassie Brilliant Blue (CBB) staining (bottom panel), and phosphorylated proteins were visualized with autoradiography (top panel). FIG. 8D is a graph plotting phosphorylation of the USP10 S76 peptide and SAMS peptide by AMPK in vitro. Initial rates of ³²P-incorporation by AMPK into the USP10 peptide and SAMS peptide were 155±36 and 95±18 pmol/min/reaction mix, respectively. The results represent the means (±SEM) of three independent experiments. FIG. 8E is a graph plotting the maximal stoichiometry of phosphorylation of the USP10 S76 peptide and SAMS peptide by AMPK. Stoichiometries of ³²P-incorporation after 30 minutes of incubation were calculated from the data shown in FIG. 8D. The results represent the means (±SEM) of three independent experiments. FIG. 8F is a picture of a gel showing phosphorylation of USP10 at Ser76 by AMPK in vitro. Bacterially expressed WT USP10 and the S76A mutant were incubated with purified AMPK in the presence of [γ-³²P]ATP and AMP. Proteins were resolved by SDS-PAGE; phosphorylated proteins were visualized with autoradiography (top), and total protein was visualized by CBB staining (bottom). FIG. 8G is a picture of immunoblots showing that AMPK phosphorylates USP10 at Ser76 in cells. USP10 expression vectors were transfected into HCT116 cells. Cells were placed in medium with or without glucose for 4 hours. FLAG-USP10 was immunoprecipitated and immunoblotted with the indicated antibodies.

FIGS. 9A-9F demonstrate that phosphorylation of USP10 promotes its activation. FIG. 9A is a graph plotting USP10 deubiquitinase activity in vitro after phosphorylation of USP10 by AMPK. Purified WT USP10 or the S76A mutant was incubated with or without active AMPK. The activities of USP10 were assayed using Ub-AMC as a substrate. FIG. 9B is a picture of an immunoblot showing that phosphorylation of USP10 regulates USP10 deubiquitinase activity in cells. Cells expressing WT or S76A USP10 were cultured in medium with or without glucose for 4 hours. Cells were lysed, and total proteins were labeled with Ub-VS probe and then subjected to immunoblotting with the indicated antibodies. FIG. 9C is a picture of immunoblots showing the effect of USP10 phosphorylation on AMPKα ubiquitination. The indicated constructs were transfected into USP10 knockdown cell lines. Cells were placed in medium without glucose for 4 hours. Cell lysates were immunoprecipitated with anti-HA beads and immunoblotted with the indicated antibodies. -Glu, glucose starvation. FIG. 9D is a picture of immunoblots showing the effect of USP10 phosphorylation on AMPKα activation. WT USP10 or S76A mutant constructs were transfected into indicated cells. Cells were placed in medium without glucose for 4 hours. Cell lysates were immunoblotted with the indicated antibodies. FIG. 9E is a graph plotting Oil-Red-O staining as a measure of lipid droplet formation in response to phosphorylation of USP10. WT USP10 or S76A mutant constructs were transfected into the indicated cells. Cells were incubated in medium supplemented with 200 μM sodium oleate overnight (20 hours) and placed in medium without glucose for 4 hours. Intracellular lipid droplet abundance was assessed by Oil-Red-O staining. The results represent the means (±SEM) of three independent experiments. **, p<0.01. FIG. 9F is a graph plotting relative mRNA levels of glycolytic genes in cells containing phosphorylated or non-phosphorylated USP10. WT USP10 or S76A mutant constructs were transfected into the indicated cells. Cells were placed in medium without glucose for 4 hours. Relative expression of aldoa, ldha, and pdkl mRNA was determined by qPCR. Transcript levels were determined relative to actin mRNA levels, and normalized relative to control cells. The results represent the means (±SEM) of three independent experiments. **, p<0.01.

FIGS. 10A-10C demonstrate gene editing in adult mouse liver by adenoviral delivery of CRISPR/Cas9. FIG. 10A is an illustration of the usp10 locus, with the shRNA and PAM sequences highlighted. Four edited sequences also are shown. FIG. 10B is a picture of a gel showing a fragment of the T7EN1 gene before and after gene editing with adenoviral CRISPR/Cas9. AdenoCas9 virus against GFP (control) or USP10 as well as the buffer in which the viruses were suspended for injection (control) were injected into 6-week old male C57BL/6 mice via tail vein. Ten days after the injection, a third of the livers from the injected animals were removed for T7EN1 gene editing analysis. FIG. 10C is a picture of Western blots analyzing USP10 levels from the indicated tissues.

FIGS. 11A-11G demonstrate that knockout of hepatic Usp10 leads to multiple metabolic defects. FIG. 11A is a picture of Western blots showing AMPK and ACC1 phosphorylation levels in liver from the indicated mice. FIG. 11B is a series of pictures showing hematoxylin & eosin (H&E) staining of liver tissue from the indicated mice. FIGS. 11C and 11D are graphs plotting liver triglyceride (FIG. 11C) and cholesterol (FIG. 11D) levels in the indicated mice. Lipids were extracted and hepatic triglyceride and cholesterol levels were assayed using a commercial kit. Values are expressed as mean±SEM. n=6. **, p<0.01. FIG. 11E is a graph plotting blood glucose levels measured in tail vein blood samples of the indicated mice using a glucometer. Values are expressed as mean±SEM (n=6). **, p<0.01. FIG. 11F is a graph plotting the glucose infusion rate in the indicated mice. Blood glucose levels were measured in tail vein blood samples using a glucometer. Values are expressed as ±SEM. n=6. **, p<0.01. FIG. 11G is a diagram of a model for the AMPK-USP10 positive feedback loop.

FIGS. 12A-12H demonstrate that USP10 interacts with and regulates mTOR signaling. FIGS. 12A and 12B are pictures of immunoblots from co-immuno-precipitation assays showing that USP10 interacts with mTOR. FIG. 12C is a picture of an immunoblot demonstrating that USP10 regulates mTOR ubiquitination. FIG. 12D is a graph plotting survival of cancer cells with and without USP10, after treatment with the mTOR inhibitor, everolimus. The inhibitor had a greater effect on cells in the absence of USP10, demonstrating that USP10 regulates the response of cancer cells to everolimus. FIG. 12E is a graph plotting survival of cancer cells with and without USP10 knockdown by shRNA, after treatment with everolimus. The mTOR inhibitor had a greater effect on the USP10 knockdown cells than on cells treated with a control shRNA. FIGS. 12F and 12G are pictures of immunoblots showing that USP10 negatively regulates mTOR signaling, as USP10 knockout (FIG. 12F) or knockdown (FIG. 12G) resulted in increased levels of phospho-S6K. FIG. 12H is a picture of an immunoblot demonstrating that USP10 negatively regulates mTOR signaling. As in FIG. 12G, USP10 knockdown using shRNA increased the level of phospho-S6K, while reconstitution of USP10 in knockdown cells resulted in decreased levels of phospho-S6K.

### DETAILED DESCRIPTION

Protein ubiquitination is an important posttranslational modification that regulates various biological functions. Ubiquitination through K48 of the ubiquitin chain generally targets proteins for degradation, whereas K63-linked ubiquitination often regulates signaling and trafficking (Clague and Urbé, Cell 143(5):682-685, 2010). As described in the Examples below, AMPKα is ubiquitinated mainly through the K63 specific ubiquitin chain. Under normal conditions, ubiquitination of AMPKα may mask it or change its structure to block access of LKB1. As further described below, under energy stress, USP10 removes the ubiquitination of AMPKα and exposes AMPKα to LKB1.

USP10 is a 798 amino-acid protein with a predicted catalytic domain between about residues 414 to 793. Representative human USP10 nucleic acid and amino acid sequences are provided below as SEQ ID NO:1 and SEQ ID NO:2, respectively. The start and stop codons in SEQ ID NO:1 are underlined; nucleotides 143-2536 encode SEQ ID NO:2. As described herein, phosphorylation of human USP10 at a single serine residue (Ser76; underlined in SEQ ID NO:2) is necessary to activate the enzyme; disruption of this phosphorylation led to decreased catalytic activity toward fluorogenic substrates and Ub-VS binding.

(SEQ ID NO: 1)
ctccccgcgccccgcggcgcgcggccagtgcgcaggcgcggcggccgatg
cgagtgtgtatgtgcgggcgagaagatggcggcggcgggggaagcagcgt
gagcagccggaggatcgcggagtcccaatgaaacgggcagccatggccct
ccacagcccgcagtatatttttggagattttagccctgatgaattcaatc
aattctttgtgactcctcgatcttcagttgagcttcctccatacagtgga
acagttctgtgtggcacacaggctgtggataaactacctgatggacaaga
atatcagagaattgagtttggtgtcgatgaagtcattgaacccagtgaca
ctttgccgagaacccccagctacagtatttcaagcacactgaaccctcag
gcccctgaatttattctcggttgtacagcttccaaaataacccctgatgg
tatcactaaagaagcaagctatggctccatcgactgccagtacccaggct
ctgccctcgctttggatggaagttctaatgtggaggcggaagttttggaa
aatgatggtgtctcaggtggtcttggacaaagggagcgtaaaaagaagaa
aaagcggccacctggatattacagctatttgaaagatggtggcgatgata
gtatctccacagaagccctggtcaatggccatgccaattcagcagtcccg
aacagtgtcagtgcagaggatgcagaatttatgggtgacatgcccccgtc
agttacgcccaggacttgtaacagcccccagaactccacagactctgtca
gtgacattgtgcctgacagtcctttccccggagcactcggcagtgacacc
aggactgcagggcagccagaggggggccccggggctgattttggtcagtc
ctgcttccctgcagaggctggcagagacaccctgtcaaggacagctgggg
ctcagccctgcgttggtaccgatactactgaaaaccttggagttgctaat
ggacaaatacttgaatcctcgggtgagggcacagctaccaacggggtgga
gttgcacaccacggaaagcatagacttggacccaaccaaacccgagagtg
catcacctcctgctgacggcacgggctctgcatcaggcacccttcctgtc
agccagcccaagtcctgggccagcctctttcatgattctaagccctcttc
ctcctcgccggtggcctatgtggaaactaagtattcccctcccgccatat
ctcccctggtttctgaaaagcaggttgaagtcaaagaagggcttgttccg
gtttcagaggatcctgtagccataaagattgcagagttgctggagaatgt
aaccctaatccataaaccagtgtcgttgcaaccccgtgggctgatcaata
aagggaactggtgctacattaatgctacactgcaggcattggttgcttgc
ccgccgatgtaccacctgatgaagttcattcctctgtattccaaagtgca
aaggccttgtacgtcaacacccatgatagacagctttgttcggctaatga
atgagttcactaatatgccagtacctccaaaaccccgacaagctcttgga
gataaaatcgtgagggatattcgccctggagctgcctttgagcccacata
tatttacagactcctgacagttaacaagtcaagcctgtctgaaaagggtc
gacaagaagatgctgaggaatacttaggcttcattctaaatggacttcat
gaggaaatgttgaacctaaagaagcttctctcaccaagtaatgaaaaact
tacgatttccaacggccccaaaaaccactcggtcaatgaagaagagcagg
aagaacaaggtgaaggaagcgaggatgaatgggaacaagtgggcccccgg
aacaagacttccgtcacccgccaggcggattttgttcagactccaatcac
cggcatttttggtggacacatcaggtctgtggtttaccagcagagttcaa
aagaatctgccactttgcagccatttttcacgttgcagttggatatccag
tcagacaagatacgcacagtccaggatgcactggagagcttggtggcaag
agaatctgtccaaggttataccacaaaaaccaaacaagaggttgagataa
gtcgaagagtgactctggaaaaactccctcctgtcctcgtgctgcacctg
aaacgattcgtttatgagaagactggtgggtgccagaagcttatcaaaaa
tattgaatatcctgtggacttggaaattagtaaagaactgctttctccag
gggttaaaaataagaattttaaatgccaccgaacctatcggctctttgca
gtggtctaccatcacggcaacagtgcgacgggcggccattacactacaga
cgtcttccagatcggtctgaatggctggctgcgcatcgatgaccagacag
tcaaggtgatcaaccagtaccaggtggtgaaaccaactgctgaacgcaca
gcctacctcctgtattaccgccgagtggacctgctgtaaaccctgtgtgc
gctgtgtgtgcgcccagtgcccgcttcgtaggacaccacctcacactcac
ttcccgcctctctttagtggctctttagagagaaactctttctccctttg
caaaaatgggctagaatgaaaaggagatgccttggggttcgtgcacaaca
cagcttctgttgactctaacttccaaatcaaaatcatttggttgaaacag
actgttgcttgattttagaaaatacacaaaaacccatatttctgaaataa
tgctgattcctgagataagaaagtggatttgatccccagtctcattgctt
agtagaataaatcctgcaccagcaacaacacttgtaaatttgtgaaaatg
aattttatctttccttaaaaaagaaattttttaatccatcacacttttct
tccctaccctttagtttttgataaatgataaaaatgagccagttatcaaa
gaagaactagttcttacttcaaaagaaaaataaacataaaaaataagttg
ctggttcctaacaggaaaaattttaataattgtactgagagaaactgctt
acgtacacattgcagatcaaatatttggagttaaaatgttagtctacata
gatgggtgattgtaactttattgccattaaaagatttcaaattgcattca
tgcttctgtgtacacataatgaaaaatgggcaaataatgaagatctctcc
ttcagtagactgtttaattctgctgtagacttctctaatgctgcgtccct
aattgtacacagtttagtgatatctaggagtataaagttgtcgcccatca
ataaaaatcacaaagttggtttaaaaaaaaaaaaaaaaaaaaaaa
(SEQ ID NO: 2)
MALHSPQYIFGDFSPDEFNQFFVTPRSSVELPPYSGTVLCGTQAVDKLPD
GQEYQRIEFGVDEVIEPSDTLPRTPSYSISSTLNPQAPEFILGCTASKIT
PDGITKEASYGSIDCQYPGSALALDGSSNVEAEVLENDGVSGGLGQRERK
KKKKRPPGYYSYLKDGGDDSISTEALVNGHANSAVPNSVSAEDAEFMGDM
PPSVTPRTCNSPQNSTDSVSDIVPDSPFPGALGSDTRTAGQPEGGPGADF
GQSCFPAEAGRDTLSRTAGAQPCVGTDTTENLGVANGQILESSGEGTATN
GVELHTTESIDLDPTKPESASPPADGTGSASGTLPVSQPKSWASLFHDSK
PSSSSPVAYVETKYSPPAISPLVSEKQVEVKEGLVPVSEDPVAIKIAELL
ENVTLIHKPVSLQPRGLINKGNWCYINATLQALVACPPMYHLMKFIPLYS
KVQRPCTSTPMIDSFVRLMNEFTNMPVPPKPRQALGDKIVRDIRPGAAFE
PTYIYRLLTVNKSSLSEKGRQEDAEEYLGFILNGLHEEMLNLKKLLSPSN
EKLTISNGPKNHSVNEEEQEEQGEGSEDEWEQVGPRNKTSVTRQADFVQT
PITGIFGGHIRSVVYQQSSKESATLQPFETLQLDIQSDKIRTVQDALESL
VARESVQGYTTKTKQEVEISRRVTLEKLPPVLVLHLKRFVYEKTGGCQKL
IKNIEYPVDLEISKELLSPGVKNKNEKCHRTYRLEAVVYHHGNSATGGHY
TTDVFQIGLNGWLRIDDQTVKVINQYQVVKPTAERTAYLLYYRRVDLL

This document relates to methods and materials involved in modulating deubiquitinases (e.g., USP10 polypeptides) and/or ubiquitinated polypeptides (e.g., kinases such as AMPK and mTOR). For example, this document provides methods and materials for increasing deubiquitinase (e.g., a USP10 polypeptide) expression or activity, methods and materials for reducing ubiquitination of polypeptides (e.g., AMPK and mTOR), methods and materials for treating disorders such as insulin resistance, type 1 or type 2 diabetes, and metabolic syndrome, and methods and materials for reducing cancer cell proliferation, increasing cancer cell apoptosis, and/or treating cancer. This document also provides methods and materials for identifying agonists or antagonists of USP10-mediated activation or inhibition of substrate polypeptides (e.g., activation of AMPK or inhibition of mTOR). Representative amino acid sequences of the human AMPKα1 and AMPKα2 polypeptides are provided in SEQ ID NOS:41 and 42, respectively:

(SEQ ID NO: 41)
MRRLSSWRKMATAEKQKHDGRVKIGHYILGDTLGVGTFGKVKVGKHELTG
HKVAVKILNRQKIRSLDVVGKIRREIQNLKLFRHPHIIKLYQVISTPSDI
FMVMEYVSGGELFDYICKNGRLDEKESRRLFQQILSGVDYCHRHMVVHRD
LKPENVLLDAHMNAKIADFGLSNMMSDGEFLRTSCGSPNYAAPEVISGRL
YAGPEVDIWSSGVILYALLCGTLPFDDDHVPTLFKKICDGIFYTPQYLNP
SVISLLKHMLQVDPMKRATIKDIREHEWFKQDLPKYLFPEDPSYSSTMID
DEALKEVCEKFECSEEEVLSCLYNRNHQDPLAVAYHLIIDNRRIMNEAKD
FYLATSPPDSFLDDHHLTRPHPERVPFLVAETPRARHTLDELNPQKSKHQ
GVRKAKWHLGIRSQSRPNDIMAEVCRAIKQLDYEWKVVNPYYLRVRRKNP
VTSTYSKMSLQLYQVDSRTYLLDFRSIDDEITEAKSGTATPQRSGSVSNY
RSCQRSDSDAEAQGKSSEVSLTSSVTSLDSSPVDLTPRPGSHTIEFFEMC
ANLIKILAQ
(SEQ ID NO: 42)
MAEKQKHDGRVKIGHYVLGDTLGVGTFGKVKIGEHQLTGHKVAVKILNRQ
KIRSLDVVGKIKREIQNLKLFRHPHIIKLYQVISTPTDFFMVMEYVSGGE
LFDYICKHGRVEEMEARRLFQQILSAVDYCHRHMVVHRDLKPENVLLDAH
MNAKIADFGLSNMMSDGEFLRTSCGSPNYAAPEVISGRLYAGPEVDIWSC
GVILYALLCGTLPFDDEHVPTLFKKIRGGVFYIPEYLNRSVATLLMHMLQ
VDPLKRATIKDIREHEWFKQDLPSYLFPEDPSYDANVIDDEAVKEVCEKF
ECTESEVMNSLYSGDPQDQLAVAYHLIIDNRRIMNQASEFYLASSPPSGS
FMDDSAMHIPPGLKPHPERMPPLIADSPKARCPLDALNTTKPKSLAVKKA
KWHLGIRSQSKPYDIMAEVYRAMKQLDFEWKVVNAYHLRVRRKNPVTGNY
VKMSLQLYLVDNRSYLLDFKSIDDEVVEQRSGSSTPQRSCSAAGLHRPRS
SFDSTTAESHSLSGSLTGSLTGSTLSSVSPRLGSHTMDFFEMCASLITTL
AR

In some embodiments, this document provides methods and materials related to treating mammals (e.g., humans) having a clinical condition such as obesity, insulin resistance, diabetes, metabolic syndrome, a neurological disorder, and cancer. Examples of mammals that can be treated as described herein include, without limitation, humans, monkeys, dogs, cats, cows, horses, pigs, rats, and mice. Examples of cancers that can be treated as described herein include, without limitation, renal cancers (e.g., renal cell carcinomas), pancreatic cancers, breast cancers, glioma, prostate cancers, lung cancers, melanoma, bladder cancers, and brain cancers such as glioma.

Treatments as described herein can be effective to reduce one or more symptoms of the clinical conditions noted herein. Symptoms of diabetes include, for example, high blood sugar levels over a prolonged period of time, frequent urination, increased thirst, and increased hunger. Symptoms of metabolic syndrome can include, without limitation, elevated triglycerides, reduced HDL cholesterol, elevated blood pressure, elevated fasting plasma glucose, microalbuminuria, and central obesity. Symptoms of insulin resistance include, for example, elevated blood sugar and elevated blood insulin.

As described herein, a clinical condition such as diabetes or cancer can be treated by increasing the level of USP10 polypeptide expression or activity. The increased level of USP10 polypeptide expression or activity can reduce ubiquitination of substrate polypeptides such as AMPK and mTOR, which, as described herein, increases the activity of AMPK but decreases the activity of mTOR. In some cases, the level of USP10 activity within cells can be increased by administering to the cells a composition containing one or more USP10 polypeptides (e.g., a USP10 polypeptide having the amino acid sequence set forth in SEQ ID NO:2, or a biologically active fragment or functional variant thereof). In some cases, the level of USP10 polypeptide expression or activity within cells can be increased by administering to the cells a composition containing a USP10 agonist, or a nucleic acid encoding a USP10 polypeptide (e.g., a nucleic acid having the sequence set forth in SEQ ID NO:1, nucleotides 143-2536 of SEQ ID NO:1, or a fragment or variant thereof). A nucleic acid can encode a full-length USP10 polypeptide, such as a human USP10 polypeptide having the amino acid sequence set forth in SEQ ID NO:2, a biologically active fragment of a USP10 polypeptide (e.g., a fragment containing amino acid residues 520 to 793 of SEQ ID NO:2), or a functional variant of a USP10 polypeptide that is catalytically active and can have activity that is the same, higher or lower than the parent protein or protein domain. In some cases, a functional variant of a USP10 polypeptide can have an amino acid sequence that is at least 95% (e.g., at least 95%, at least 96%, at least 97%, at least 98%, or at least 99%) identical to the sequence set forth in SEQ ID NO:2. In some cases, a functional variant of a USP10 polypeptide can be encoded by a nucleic acid having a sequence that is at least 95% (e.g., at least 95%, at least 96%, at least 97%, at least 98%, or at least 99%) identical to the nucleic acid sequence set forth in SEQ ID NO:1, or at least 95% (e.g., at least 95%, at least 96%, at least 97%, at least 98%, or at least 99%) identical to nucleotides 143-2536 of SEQ ID NO:1.

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -l; -r is set to 2; and all other options are left at their default setting. For example, the following command can be used to generate an output file containing a comparison between two sequences: C:\Bl2seq c:\seq1.txt -j c:\seq2.txt -p blastn -o c:\output.txt -q -l -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence (e.g., SEQ ID NO:2), or by an articulated length (e.g., 100 consecutive nucleotides or amino acid residues from a sequence set forth in an identified sequence), followed by multiplying the resulting value by 100. For example, a nucleic acid sequence that has 760 matches when aligned with the sequence set forth in SEQ ID NO:2 is 95.2 percent identical to the sequence set forth in SEQ ID NO:2 (i.e., 760÷798×100=95.2). It is noted that the percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. It also is noted that the length value will always be an integer.

The terms “nucleic acid” and “polynucleotide” are used interchangeably, and refer to both RNA and DNA, including cDNA, genomic DNA, synthetic (e.g., chemically synthesized) DNA, and DNA (or RNA) containing nucleic acid analogs. Polynucleotides can have any three-dimensional structure. A nucleic acid can be double-stranded or single-stranded (i.e., a sense strand or an antisense single strand). Non-limiting examples of polynucleotides include genes, gene fragments, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

As used herein, “isolated,” when in reference to a nucleic acid, refers to a nucleic acid that is separated from other nucleic acids that are present in a genome, e.g., a plant genome, including nucleic acids that normally flank one or both sides of the nucleic acid in the genome. The term “isolated” as used herein with respect to nucleic acids also includes any non-naturally-occurring sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences, as well as DNA that is incorporated into a vector, an autonomously replicating plasmid, a virus (e.g., a pararetrovirus, a retrovirus, lentivirus, adenovirus, or herpes virus), or the genomic DNA of a prokaryote or eukaryote. In addition, an isolated nucleic acid can include a recombinant nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

A nucleic acid can be made by, for example, chemical synthesis or polymerase chain reaction (PCR). PCR refers to a procedure or technique in which target nucleic acids are amplified. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

The term “polypeptide” as used herein refers to a compound of two or more subunit amino acids regardless of post-translational modification (e.g., phosphorylation or glycosylation). The subunits may be linked by peptide bonds or other bonds such as, for example, ester or ether bonds. The term “amino acid” refers to either natural and/or unnatural or synthetic amino acids, including D/L optical isomers.

By “isolated” or “purified” with respect to a polypeptide it is meant that the polypeptide is separated to some extent from the cellular components with which it is normally found in nature (e.g., other polypeptides, lipids, carbohydrates, and nucleic acids). A purified polypeptide can yield a single major band on a non-reducing polyacrylamide gel. A purified polypeptide can be at least about 75% pure (e.g., at least 80%, 85%, 90%, 95%, 97%, 98%, 99%, or 100% pure). Purified polypeptides can be obtained by, for example, extraction from a natural source, by chemical synthesis, or by recombinant production in a host cell or transgenic plant, and can be purified using, for example, affinity chromatography, immunoprecipitation, size exclusion chromatography, and ion exchange chromatography. The extent of purification can be measured using any appropriate method, including, without limitation, column chromatography, polyacrylamide gel electrophoresis, or high-performance liquid chromatography.

A USP10 polypeptide or a nucleic acid encoding a USP10 polypeptide or fragment thereof can be administered to a mammal using any appropriate method. For example, a nucleic acid can be administered to a mammal using a vector such as a viral vector. A “vector” is a nucleic acid molecule capable of transporting another nucleic acid to which it has been linked. In particular, a “vector” is a replicon, such as a plasmid, phage, or cosmid, into which another DNA segment may be inserted so as to bring about the replication of the inserted segment. A vector can be, without limitation, a viral vector, a plasmid, a RNA vector or a linear or circular DNA or RNA molecule which can consist of chromosomal, non-chromosomal, semi-synthetic, or synthetic nucleic acids. Vectors can be capable of autonomous replication (episomal vector) and/or expression of nucleic acids to which they are linked (expression vectors). Large numbers of suitable vectors are known to those of skill in the art and commercially available.

Viral vectors for administering nucleic acids (e.g., a nucleic acid encoding a USP10 polypeptide or fragment thereof) to a mammal are known in the art and can be prepared using standard materials (e.g., packaging cell lines, helper viruses, and vector constructs). See, for example, Gene Therapy Protocols (Methods in Molecular Medicine), ed. Jeffrey R. Morgan, Humana Press, Totowa, N.J. (2002) and Viral Vectors for Gene Therapy: Methods and Protocols, ed. Curtis A. Machida, Humana Press, Totowa, N.J. (2003). Virus-based nucleic acid delivery vectors are typically derived from animal viruses, such as adenoviruses, adeno-associated viruses, retroviruses, lentiviruses, vaccinia viruses, herpes viruses, and papilloma viruses. Lentiviruses are a genus of retroviruses that can be used to infect cells (e.g., cancer cells). Adenoviruses contain a linear double-stranded DNA genome that can be engineered to inactivate the ability of the virus to replicate in the normal lytic life cycle. Adenoviruses and adeno-associated viruses can be used to infect cancer cells.

Viral vectors for nucleic acid delivery can be genetically modified such that the pathogenicity of the virus is altered or removed. The genome of a virus can be modified to increase infectivity and/or to accommodate packaging of a nucleic acid, such as a nucleic acid encoding a USP10 polypeptide or fragment thereof. A viral vector can be replication-competent or replication-defective, and can contain fewer viral genes than a corresponding wild-type virus or no viral genes at all.

In addition to nucleic acid encoding a USP10 polypeptide or fragment thereof, a viral vector can contain regulatory elements operably linked to a nucleic acid encoding a USP10 polypeptide or fragment thereof. Such regulatory elements can include promoter sequences, enhancer sequences, response elements, signal peptides, internal ribosome entry sequences, polyadenylation signals, terminators, or inducible elements that modulate expression (e.g., transcription or translation) of a nucleic acid. The choice of element(s) that may be included in a viral vector depends on several factors, including, without limitation, inducibility, targeting, and the level of expression desired. For example, a promoter can be included in a viral vector to facilitate transcription of a nucleic acid encoding a USP10 polypeptide or fragment thereof. A promoter can be constitutive or inducible (e.g., in the presence of tetracycline), and can affect the expression of a nucleic acid encoding a USP10 polypeptide or fragment thereof in a general or tissue-specific manner. Tissue-specific promoters include, without limitation, enolase promoter, prion protein (PrP) promoter, and tyrosine hydroxylase promoter.

As used herein, “operably linked” refers to positioning of a regulatory element in a vector relative to a nucleic acid in such a way as to permit or facilitate expression of the encoded polypeptide. For example, a viral vector can contain a neuronal-specific enolase promoter and a nucleic acid encoding a USP10 polypeptide or fragment thereof. In this case, the enolase promoter is operably linked to a nucleic acid encoding a USP10 polypeptide or fragment thereof such that it drives transcription in neuronal tumor cells.

A nucleic acid encoding a USP10 polypeptide or fragment thereof also can be administered to cells using non-viral vectors. Methods of using non-viral vectors for nucleic acid delivery are known to those of ordinary skill in the art. See, for example, Gene Therapy Protocols (Methods in Molecular Medicine), ed. Jeffrey R. Morgan, Humana Press, Totowa, N.J. (2002). For example, a nucleic acid encoding a USP10 polypeptide or fragment thereof can be administered to a mammal by direct injection (e.g., an intratumoral injection) of nucleic acid molecules (e.g., plasmids) comprising nucleic acid encoding a USP10 polypeptide or fragment thereof, or by administering nucleic acid molecules complexed with lipids, polymers, or nanospheres.

A nucleic acid encoding a USP10 polypeptide or fragment thereof can be produced by standard techniques, including, without limitation, common molecular cloning, polymerase chain reaction (PCR), chemical nucleic acid synthesis techniques, and combinations of such techniques. For example, PCR or RT-PCR can be used with oligonucleotide primers designed to amplify nucleic acid (e.g., genomic DNA or RNA) encoding a USP10 polypeptide or fragment thereof.

In some cases, a nucleic acid encoding a USP10 polypeptide or fragment thereof can be isolated from a healthy mammal or from a mammal having a clinical condition such as obesity, insulin resistance, diabetes (e.g., type 1 or type 2 diabetes), metabolic syndrome, a neurological disorder, or cancer. For example, a nucleic acid that encodes a wild type USP10 polypeptide having the amino acid sequence set forth in SEQ ID NO:2 can be isolated from a human containing that nucleic acid. The isolated nucleic acid can then be used to generate a viral vector, for example, which can be administered to a mammal so that the level of a USP10 polypeptide or fragment thereof in cancer cells within the mammal is increased.

This document also provides methods and materials related to identifying agonists or antagonists of USP10-mediated action on polypeptides such as AMPK and mTOR. For example, this document provides methods and materials for using USP10 and AMPK or mTOR polypeptides (e.g., ubiquitinated AMPK or mTOR polypeptides) to identify agents that increase or decrease the ability of USP10 to deubiquitinate substrates such as AMPK and mTOR. In some cases, the kinase activity of ubiquitinated AMPK or mTOR treated with USP10 in the presence and absence of a test agent can be assessed to determine whether or not the test agent increases or decreases the activity of the kinases. For example, an agent that increases the activity of a ubiquitinated AMPK polypeptide or decreases the activity of a ubiquitinated mTOR polypeptide in a manner dependent on the USP10 polypeptide can be considered an agonist of USP10 polypeptide-mediated activation of AMPK polypeptides and inhibition of mTOR polypeptides. Conversely, an agent that decreases the activity of a ubiquitinated AMPK polypeptide or increases the activity of a ubiquitinated mTOR polypeptide in a manner dependent on the USP10 polypeptide can be considered an antagonist of USP10 polypeptide-mediated activation of AMPK polypeptides and inhibition of mTOR polypeptides. The activity of kinase polypeptides can be assessed using assays that are known in the art, including those described in the Examples herein.

USP10 polypeptide agonists and antagonists can be identified by screening test agents (e.g., from synthetic compound libraries and/or natural product libraries). Test agents can be obtained from any commercial source, or can be chemically synthesized using methods that are known to those of skill in the art. Test agents can be screened and characterized using in vitro cell-based assays, cell free assays, and/or in vivo animal models.

USP10 agonists or antagonists also can be identified using an in vitro screen that includes using purified His-tagged USP10 polypeptide together with ubiquitin-AMC (BIOMOL) as the substrate. Ubiquitin-AMC is a fluorogenic substrate for a wide range of deubiquitinating enzymes (Dang et al., Biochemistry, 37:1868 (1998)). This fluorescence can allow high-throughput screen of USP10 agonists and antagonists in vitro. Further, in some embodiments, agents that act as USP10 agonists or antagonists (e.g., modified ubiquitin molecules) can be identified using conformational display assays as described, for example, in U.S. Pat. No. 9,139,863 (see, also, Zhang et al., Nature Chem Biol 9:51-58, 2013).

The invention will be further described in the following examples, which do not limit the scope of the invention described in the claims.

### EXAMPLES

### Example 1

### Materials and Methods

Experimental animals—CRISPR/Cas9-mediated gene editing was performed in adult live mice to obtain liver specific USP10 gene knockout directly, without resorting to knockout in the germline as described elsewhere (Cheng et al., Febs Lett 588:3954-3958, 2014). Briefly, AdenoCas9 virus against GFP (control) or USP10 were packaged in 293A cells. The viral particles were concentrated and purified through gradient centrifugation. AdenoCas9n targeting USP10 (1×10¹⁰ virus particles/animal), as well as control virus and a control containing only the buffer in which the viruses were suspended, was injected into mice via the tail vein. The vast majority of adenoviruses injected into the tail vein end up in the liver, and other organs and tissues are spared. Ten days after the injection, the animals were sacrificed, the liver was isolated, and genomic DNA, total RNA, and proteins were extracted for analyses.

Measurement of intracellular lipid, cholesterol, and triglyceride contents—Intracellular lipid droplet contents of cultured cells were evaluated by Oil Red O staining. Lipids in mouse liver were extracted as described elsewhere (Folch et al., J Biol Chem 226:497-509, 1957). Cholesterol and triglyceride levels in extracted lipids were measured enzymatically using reagents from Cayman Chemical (Ann Arbor, Mich.) according to the manufacturer's instruction.

Hyperinsulinemic-euglycemic clamp—Hyperinsulinemic-euglycemic clamps were performed as described elsewhere (Katz et al., J Clin Endocr Metab 85:2402-2410, 2000). Briefly, mice were anesthetized by intraperitoneal injection of ketamine (100 mg/kg) and xylazine (20 mg/kg). Heparin (200 U/mL) was intraperitoneally injected to prevent blood clotting. A polyethylene catheter was inserted into the right jugular vein for infusion of 20% glucose and insulin (2 mU/mL) in normal saline, using a peristaltic pump. The left carotid artery was cannulated for blood sampling. Insulin (3 mU/kg/min) was infused through the jugular vein catheter from 0 to 120 min. During this period, the blood glucose concentration was monitored every 5 minutes with a glucometer and clamped at euglycemic levels (5.0±0.5 mmol/L) by a variable infusion of 20% glucose. Clamping was achieved by 90 minutes and maintained for 30 minutes. Mean glucose infusion rates (ml/min/kg) were calculated during the last 30 minutes of the clamp. To determine blood glucose levels, mice were fasted overnight and their blood glucose was monitored by applying tail blood to the glucometer.

Cell culture—HCT116 p53^{+/+} and p53^{-/-}cells were cultured in McCoy's 5A supplemented with 10% FBS. 293T, AMPKα1/2 DKO MEFs were cultured in DMEM supplemented with 10% FBS. HepG2 was cultured in EMEM supplemented with 10% FBS.

Ub-VS labeling—Cells were treated as indicated, harvested, and lysed in lysis buffer (50 mM N-2-hydroxyethylpiperazineN9-2-ethanesulfonic acid; pH 7.4], 250 mM sucrose, 10 mM MgCl₂, 2 mM adenosine triphosphate, 1 mM dithiothreitol), followed by removal of debris by centrifugation. Total protein (25 mg) was labeled with 1 μM ubiquitin-vinyl sulfone (Ub-VS; U-505; Boston Biochem) probe for 30 minutes at 37° C. and then subjected to western blotting.

In vitro deubiquitinase enzymatic assay—In vitro enzymatic assays using ubiquitin-7-amido-4-methylcoumarin (Ub-AMC; U-550; Boston Biochem) were performed in 50-100 μl reaction buffer (20 mM HEPES-KOH [pH 7.8], 20 mM NaCl, 0.1 mg/ml ovalbumin [A7641; Sigma], 0.5 mM EDTA, and 10 mM DTT) at 25° C. Fluorescence was monitored in an INFINITE® M1000 PRO Fluorometer (TECAN).

Quantitative Real-Time PCR—Total mRNA was isolated from cells using a PARIS™ Kit (ThermoFisher). Quantitative PCR (qPCR) was performed using the one-step, Brilliant SYBR Green qRT-PCR master mix kit (Stratagene), and the Stratagene Mx3005P Real-Time PCR detection system (Stratagene) using primers against aldoA, ldha, and pdkl. All experiments were performed in triplicate with beta-actin as an internal control. All samples were normalized to β-actin mRNA levels. Primer sequences are shown in TABLE 1.

TABLE 1
qPCR primers

| Gene | Direction | Sequence | SEQ ID NO: |
|---|---|---|---|
| Mouse β-actin | Forward | CGGTTCCGATGCCCTGAGGCTCTT | 3 |
| | Reverse | CGTCACACTTCATGATGGAATTGA | 4 |
| Mouse aldoa | Forward | GTGGGAAGAAGGAGAACCTG | 5 |
| | Reverse | CTGGAGTGTTGATGGAGCAG | 6 |
| Mouse ldha | Forward | TGTCTCCAGCAAAGACTACTGT | 7 |
| | Reverse | GACTGTACTTGACAATGTTGGGA | 8 |
| Mouse pdk1 | Forward | ACAAGGAGAGCTTCGGGGTGGATC | 9 |
| | Reverse | CCACGTCGCAGTTTGGATTTATGC | 10 |
| human β-actin | Forward | AGGCACCAGGGCGTGAT | 11 |
| | Reverse | CGTCACACTTCATGATGGAATTGA | 12 |
| human aldoc | Forward | GCCCGTTATGCCAGTATCT | 13 |
| | Reverse | AGCCAAGACCTTCTCTGTAA | 14 |
| human ldha | Forward | GGTTGAGAGTGCTTATGA | 15 |
| | Reverse | AACACTAAGGAAGACATCA | 16 |
| human pdk1 | Forward | CGGATCAGAAACCGACACA | 17 |
| | Reverse | ACTGAACATTCTGGCTGGTGA | 18 |

Plasmids and antibodies—AMPK α1 and α2 were cloned into the pMSCV-puro (Sigma), pCMV-HA, pIRES-SFB, pLenti-FLAG 6.3, and pLVX3-IRESpuro vectors for mammalian expression. USP10 expression and shRNA plasmids were as described elsewhere (Yuan et al., Cell 140:384-U121, 2010). Point mutations were performed by a PCR-based site-directed mutagenesis method using Pfu polymerase (Stratagene). AMPK α1, AMPK α2, AMPK β1, AMPK β2, AMPK γ1 were cloned into pGEX-4T-1 for bacterial expression. HA-tagged ubiquitin and ubiquitin lysine mutants were obtained from Addgene. Antibodies against AMPKα1, AMPKα2, pAMPK T172, LKB1 and pan-AMPK substrates were purchased from Cell Signaling. Ubiquitin antibody was obtained from Santa Cruz. Anti-FLAG (M2), anti-HA, and anti-β-actin antibodies were purchased from Sigma.

Lentivirus Infection—Lentiviruses for infection of HCT116, HepG2, and MEF cells were packaged in HEK293T cells using LIPOFECTAMINE® 2000 (ThermoFisher) transfection. Forty-eight hours after transfection, medium was collected and added to the target cells with 8 μg/mL polybrene to enhance infection efficiency.

In vitro deubiquitination of AMPKα—To prepare ubiquitinated AMPKα as the substrate for the in vitro deubiquitination assay, HCT116 cells were transfected with both HA-Ub and FLAG-AMPKα. After 36 hours, ubiquitinated AMPK was purified from the cell extracts with anti-FLAG-affinity column in FLAG-lysis buffer (50 mM Tris-HCl pH 7.8, 137 mM NaCl, 10 mM NaF, 1 mM EDTA, 1% Triton X-100, 0.2% Sarcosyl, 1 mM DTT, 10% glycerol, and fresh proteinase inhibitors). Following extensive washing with the FLAG-lysis buffer, proteins were eluted with FLAG-peptides (Sigma). The recombinant His-USP10 and USP10CA polypeptides were expressed in BL21 cells and purified with the His-tag purification column (Novagen). For the in vitro deubiquitination assay, ubiquitinated AMPK protein was incubated with recombinant USP10 in a deubiquitination buffer (50 mM Tris-HCl pH 8.0, 50 mM NaCl, 1 mM EDTA, 10 mM DTT, 5% glycerol) overnight at 16° C.

Denaturing Ni-NTA pull-down—Transiently transfected or virus infected cells were harvested and pellets were washed once in PBS. Cells were lysed in 8 M urea, 0.1 M NaH₂PO₄, 300 mM NaCl, 0.01 M Tris (pH 8.0). Lysates were briefly sonicated to shear DNA and incubated with Ni-NTA agarose beads (QIAGEN) for 1-2 hours at room temperature. Beads were washed 5 times with 8 M urea, 0.1 M NaH₂PO₄, 300 mM NaCl, 0.01 M Tris (pH 8.0). Input and beads were boiled in loading buffer and subjected to SDS-PAGE and immunoblotting.

Denaturing immunoprecipitation for ubiquitination—Cells were lysed in 100 μl 62.5 mM Tris-HCl (PH 6.8), 2% SDS, 10% glycerol, 20 mM NEM and 1 mM iodoacetamide, boiled for 15 minutes, diluted 10 times with NETN buffer containing protease inhibitors, 20 mM NEM, and 1 mM iodoacetamide, and centrifuged to remove cell debris. Cell extracts were subjected to immunoprecipitation with the indicated antibodies, and blotted as indicated.

Immunoprecipitation, immunoblotting, and in vitro pull-down assay—Cell lysates were prepared and immunoprecipitation and immunoblotting were performed as described elsewhere (Yuan et al., supra). In brief, cells were lysed with NETN buffer (20 mM Tris-HCl, pH 8.0, 100 mM NaCl, 1 mM EDTA, 0.5% Nonidet P-40) containing 50 mM β-glycerophosphate, 10 mM NaF, and 1 mg/ml each of pepstatin A and aprotinin. Whole cell lysates were centrifuged at 12000 rpm for 15 minutes, and then incubated with 2 μg of antibody and protein A or protein G Sepharose beads (Amersham Biosciences) for 2 hours or overnight at 4° C. Immunocomplexes were then washed with NETN buffer three times and separated by SDS-PAGE. Immunoblotting was performed following standard procedures. GST fusion proteins were bound to glutathione-Sepharose for 3 hours at 4° C. The beads were washed with PBS twice and incubated with the indicated proteins for 1 hour at 4° C. After washing with NETN three times, the bound proteins were separated by SDS-PAGE and immunoblotted with the indicated antibodies.

AMPK in vitro kinase assay—Purified AMPK (Upstate Biotechnology) was incubated with various substrates (0.5 μg) in kinase reaction buffer (15 mM HEPES, pH 7.0, 1 mm dithiothreitol, 18.75 mM MgCl₂, 1.25 mM EGTA, and 125 μM ATP and 12.5 μCi of radiolabeled ATP, with or without 150 μM AMP, at 30° C. for 30 minutes. The product was separated by SDS-PAGE and subjected to autoradiography. Phosphorylation was detected by incorporation of radiolabeled [γ-32P] ATP. To study the stoichiometry of phosphorylation of the USP10 S76 [TLPRTPSYSISST (SEQ ID NO:19), synthesized by Peptide 2.0] and SAMS (Abcam) peptides by AMPK, the peptides (20 μM) were individually incubated in 100 μl of reaction buffer at 30° C. Aliquots (10 μl) were removed at various times to measure ³²P incorporation.

Oil-Red-O staining—HepG2 cells were washed with ice-cold PBS, fixed with 10% formalin for 60 minutes, washed with 60% isopropanol, and stained with Oil-Red-O working solution (1.8 mg/ml of Oil-Red-O in 6:4 isopropanol: water solution) for 10 minutes at room temperature. After staining, cells were washed with water four times to remove any remaining dye. For quantification of Oil-Red-O staining, the cell-retained dye was extracted by isopropanol and the content was measured spectrophotometrically at 500 nm.

Statistics—Data are expressed as mean±standard errors of the mean (SEM). Statistical analyses were performed with the Student's t-test or ANOVA. Statistical significance is represented in the figures by * (p<0.05) and ** (p<0.01).

### Example 2

### USP10 Regulates AMPK Activation and Cellular Metabolism

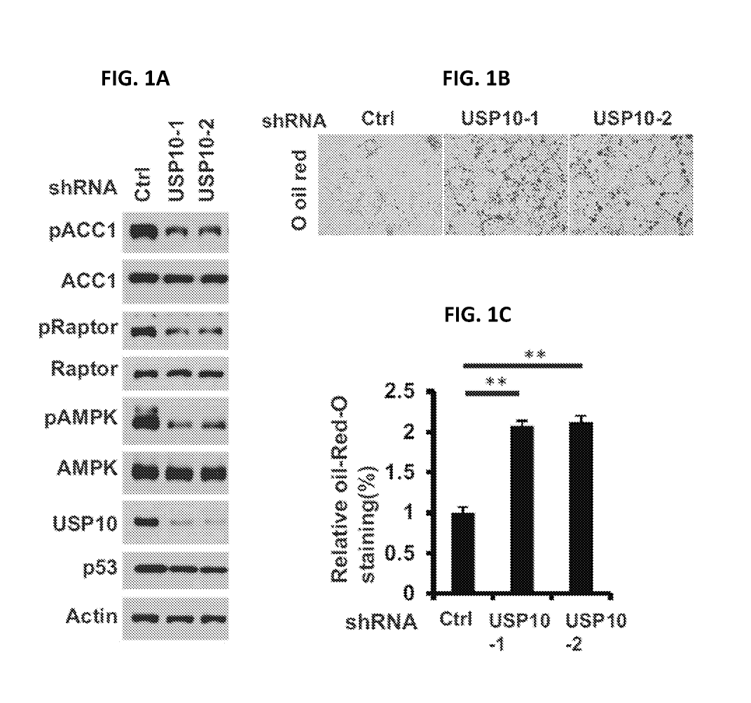

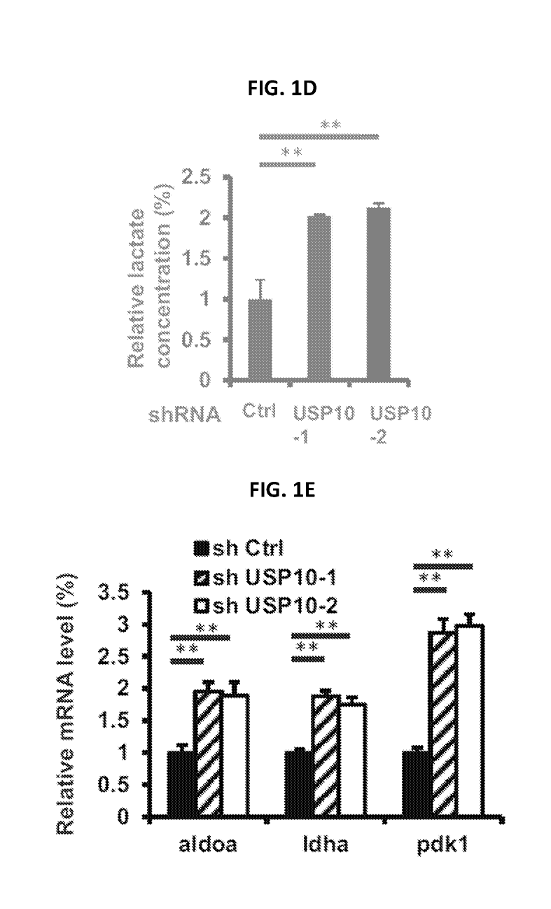

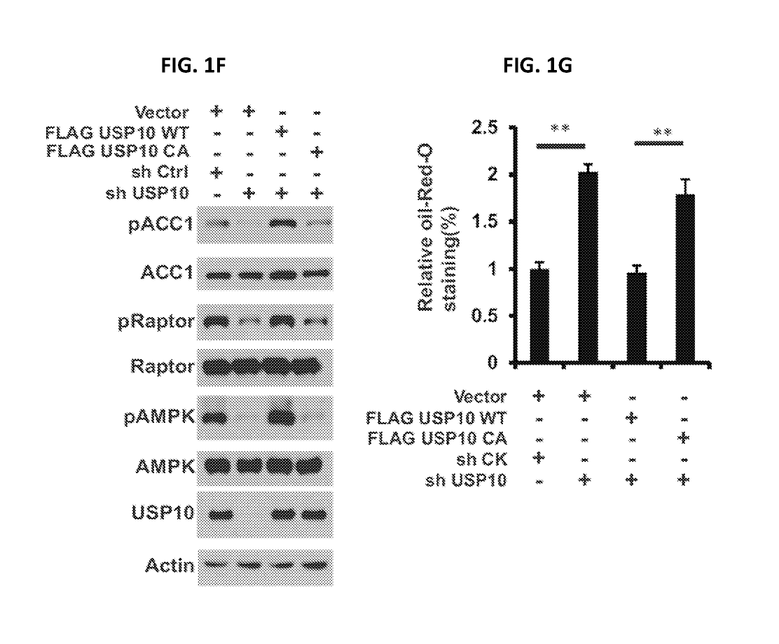

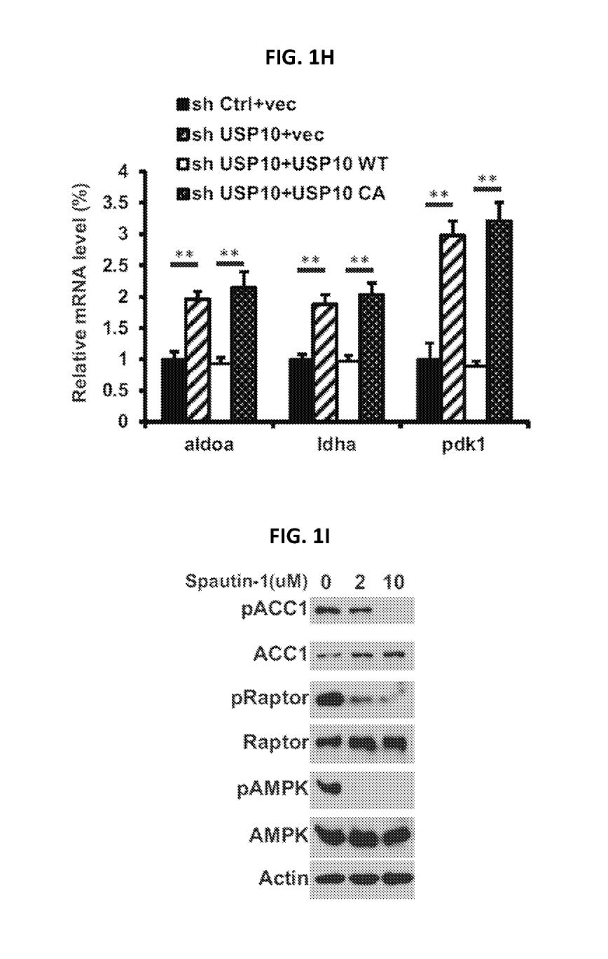

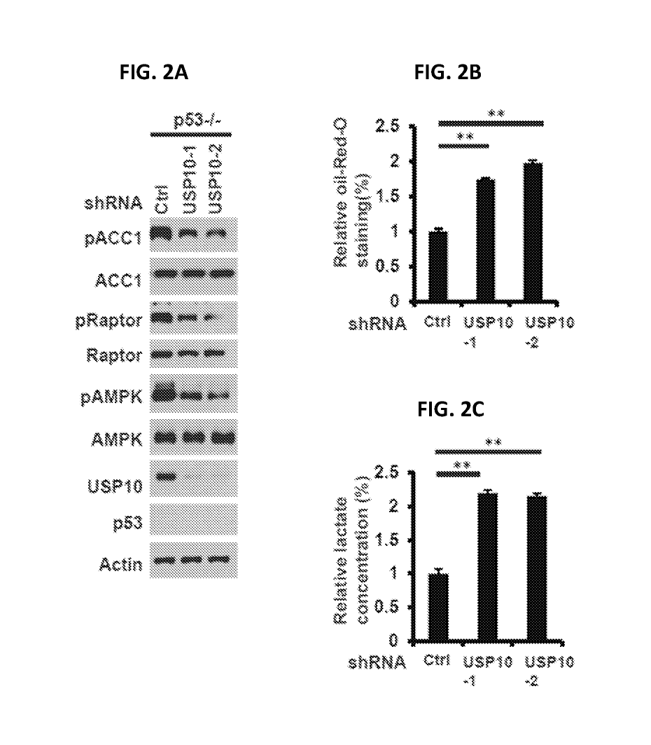

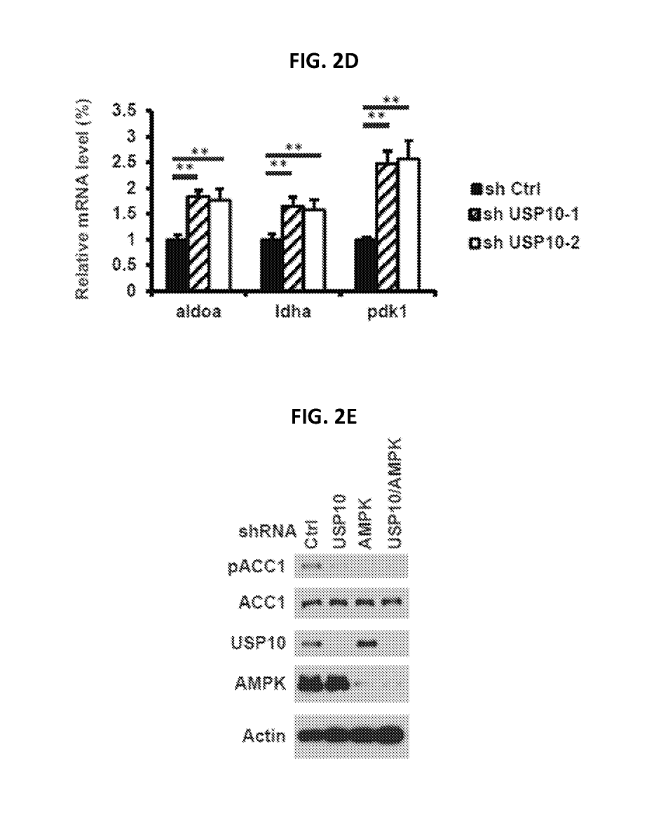

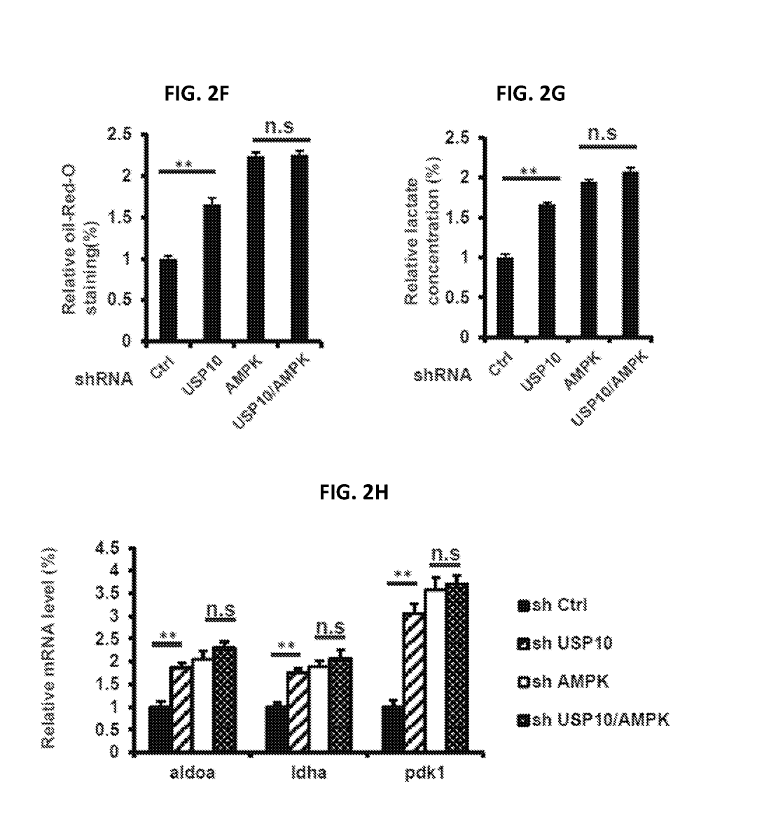

Studies described elsewhere identified USP10 as a deubiquitinase for p53, and showed that its deficiency contributes to cell transformation and cell proliferation (Yuan et al., supra). The follow-up studies discussed herein unexpectedly revealed that USP10 also regulates the AMPK pathway. For example, studies demonstrated that in USP10-deficient cells, the phosphorylation of AMPKα is dramatically decreased (FIG. 1A). Phosphorylation of the AMPK substrates, ACC1 and Raptor, also was dramatically decreased. These effects were not caused by decreased p53 levels, as similar phenotypes also were observed in HCT116 p53^{-/-} cells (FIG. 2A). As AMPK plays key roles in cellular metabolism such as lipid metabolism and glycolysis (Assifi et al., Am J Physiol-Endoc M 289:E794-E800, 2005; Faubert et al., Cell Metab 17:113-124, 2013; Steinberg and Kemp, Physiol Rev 89:1025-1078, 2009), studies were conducted to determine whether USP10 regulates these metabolic processes. Knocking down USP10 resulted in a significant increase in lipid drop formation (FIGS. 1B, 1C, and 2B), lactate production (FIGS. 1D and 2C), and glycolytic gene expression in p53-profient and p53-deficient cells (FIGS. 1E and 2D). All of these metabolic changes suggested that the AMPK pathway is inactivated with USP10 deficiency.

To determine the contribution of AMPK to the metabolic phenotype observed in USP10 deficient cells, RNAi was used to stably silence USP10 in control and AMPKα-deficient cells (FIG. 2E). Expression of USP10 shRNA increased lipid droplet content, lactate production, and glycolytic gene expression in control cells but not in AMPKα-deficient cells (FIGS. 2F, 2G, and 2H), indicating that USP10 regulates cellular metabolism through AMPK.

To determine whether the deubiquitinase activity of USP10 is required for its function in AMPK activation, wide type (WT) or catalytic inactive mutant (CA) USP10 was reconstructed in USP10 deficient cells. The WT USP10, but not the CA mutant, rescued AMPK from the inactivation triggered by USP10 RNAi (FIG. 1F), suggesting that USP10 deubiquitinase activity is required for AMPK activation. It was then determined whether reconstitution of WT USP10 or the CA mutant could reverse the metabolic defects triggered by loss of USP10. While reconstituted WT USP10 decreased lipid droplet formation and glycolytic gene expression, expression of the CA mutant had no significant effect (FIGS. 1G and 1H). Further, after inhibiting USP10 activity with a small molecular inhibitor of USP10 (Spautin-1; see, Liu et al., Cell 147:223-234, 2011), the AMPK activation was largely inhibited (FIG. 1I). These results suggested that USP10 deubiquitinase activity is required for its effects on AMPK activation and cellular metabolism.

### Example 3

### USP10 Interacts with AMPK and Deubiquitinates AMPKα

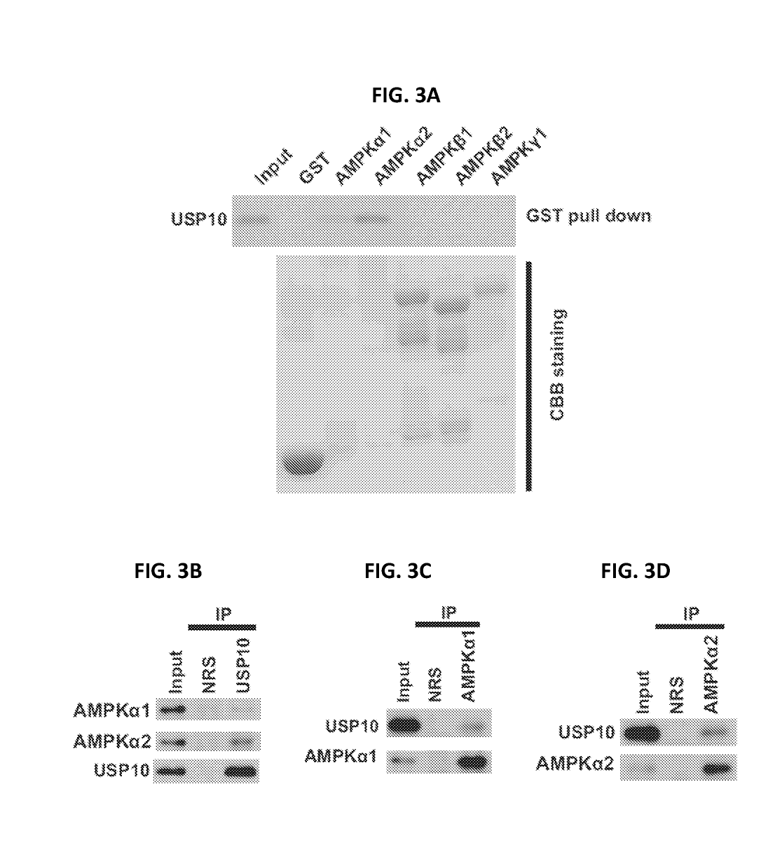

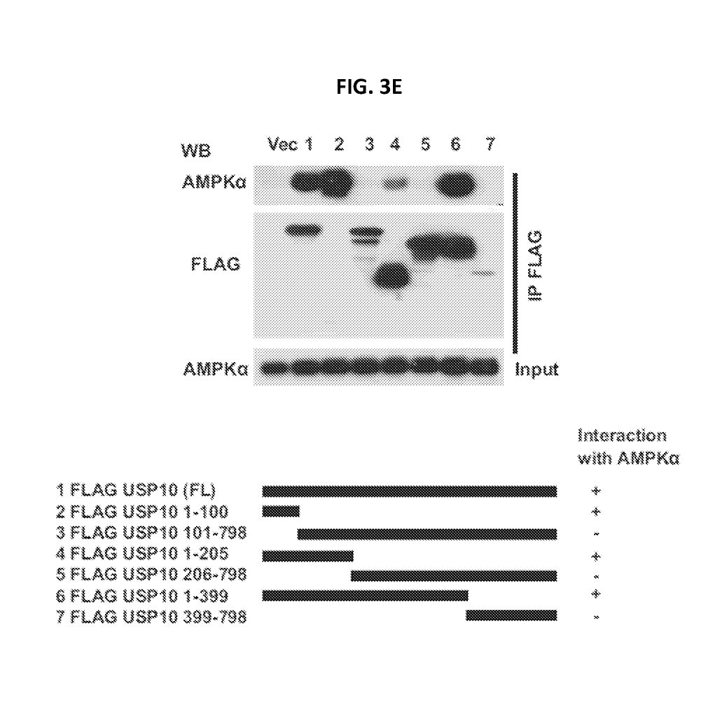

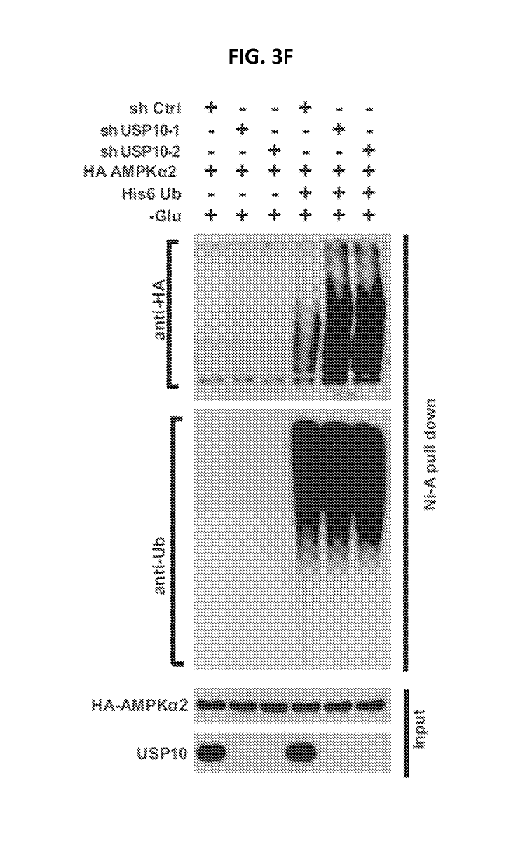

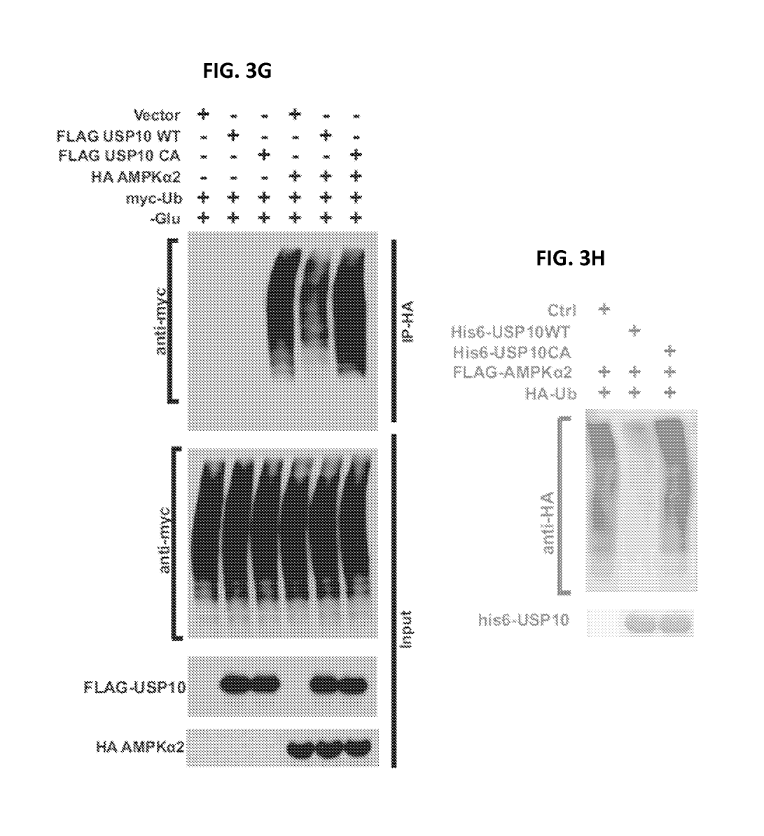

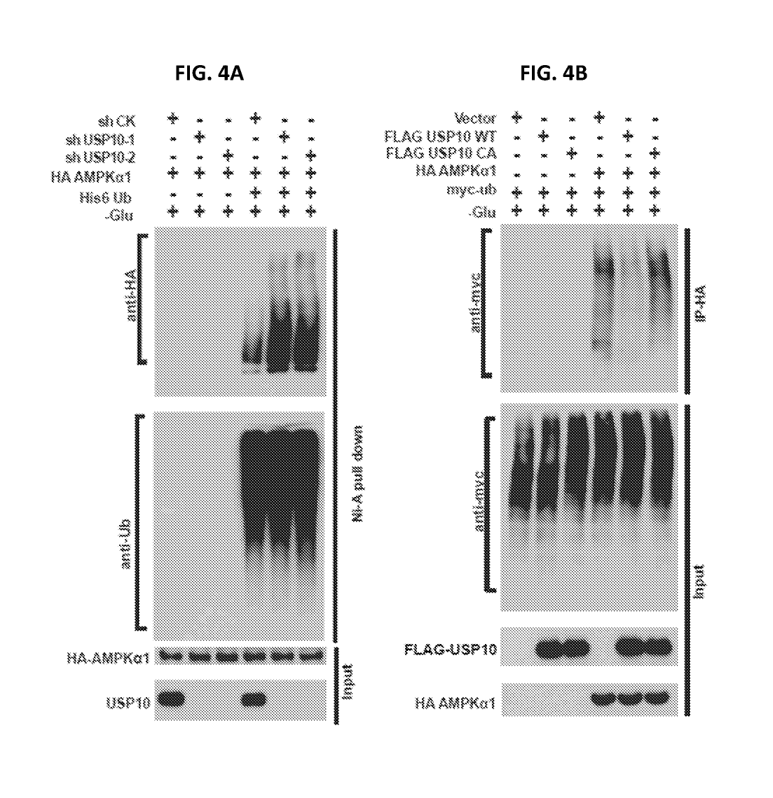

The mechanism by which USP10 modulates AMPK activation was then explored. Using recombinant proteins, it was found that USP10 directly interacts with AMPK α1 and α2 but not the other AMPK subunits (FIG. 3A). The physical interaction in cells was further confirmed by coimmunoprecipitation (Co-IP) assays, demonstrating that AMPKα Co-IPed with USP10 (FIGS. 3B, 3C, and 3D). Mapping the region of USP10 required for AMPKα binding revealed that the N-terminal region (amino acids 1-100) is critical for the interaction with AMPKα (FIG. 3E).

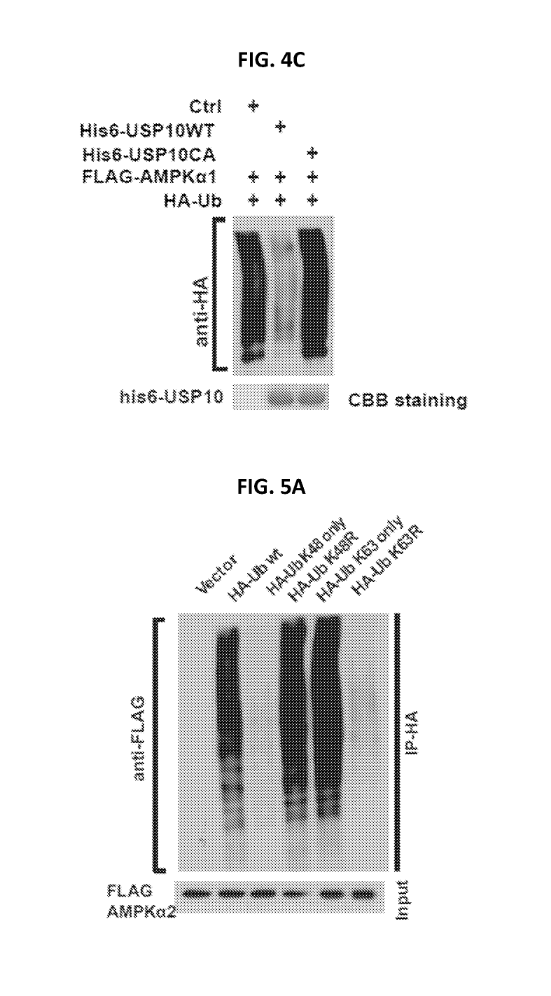

As USP10 is a deubiquitinase that binds to AMPKα and regulates AMPK activation, it was hypothesized that USP10 might regulate AMPK activation through the deubiquitination of AMPKα. When USP10 was knocked down, the ubiquitination of AMPKα significantly increased (FIGS. 3F and 4A). WT USP10 or the catalytically inactive mutant were further expressed in USP10 deficient cells, revealing that WT USP10, but not the CA mutant, decreased ubiquitination of AMPKα (FIGS. 3G and 4B), suggesting that USP10 regulates AMPKα ubiquitination in cells. To further confirm the deubiquitinase activity toward AMPKα, an in vitro deubiquitination assay was performed using bacterially-expressed USP10. While WT USP10 removed ubiquitin chain from AMPKα, the catalytically inactive CA mutant could not (FIGS. 3H and 4C). These results indicated that USP10 is a deubiquitinase for AMPKα.

### Example 4

### Ubiquitination of AMPKα Regulates its Activation

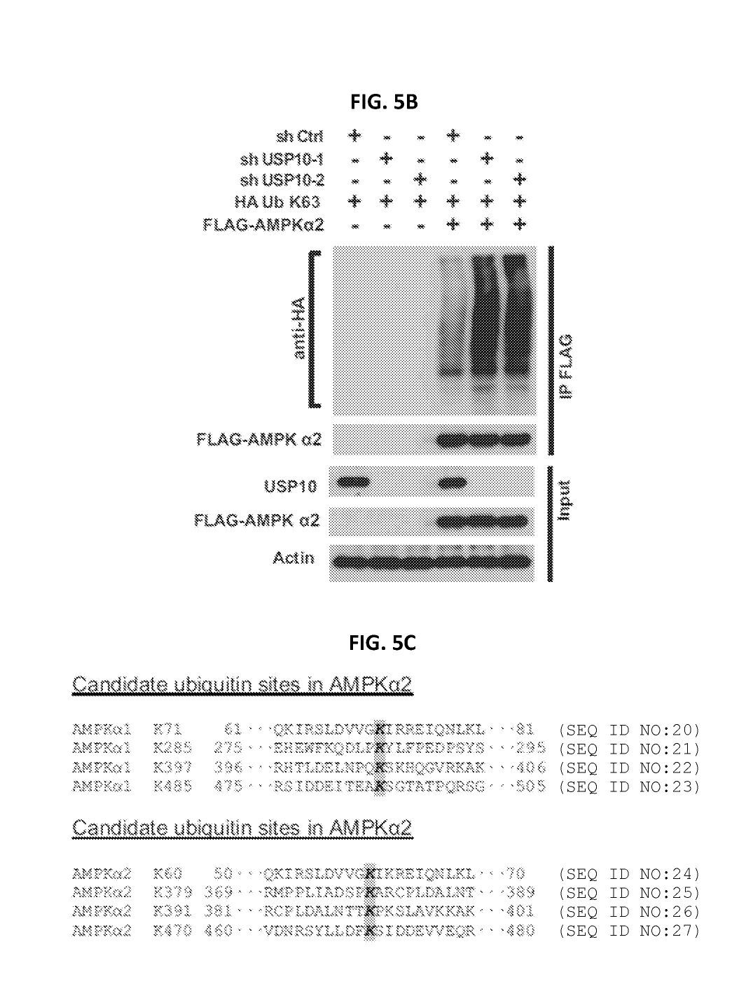

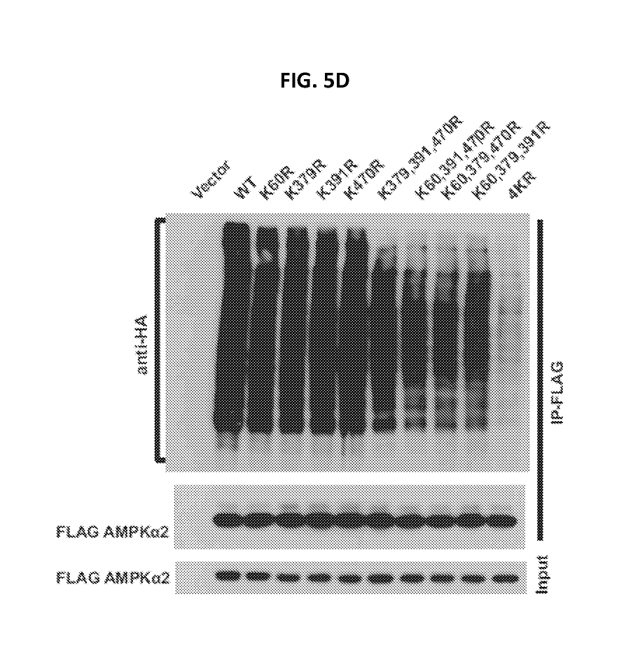

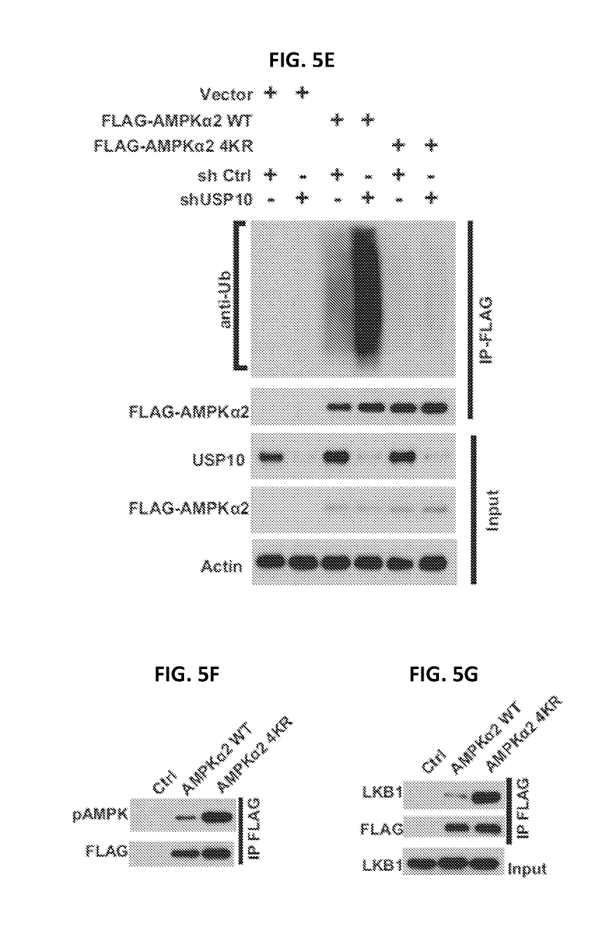

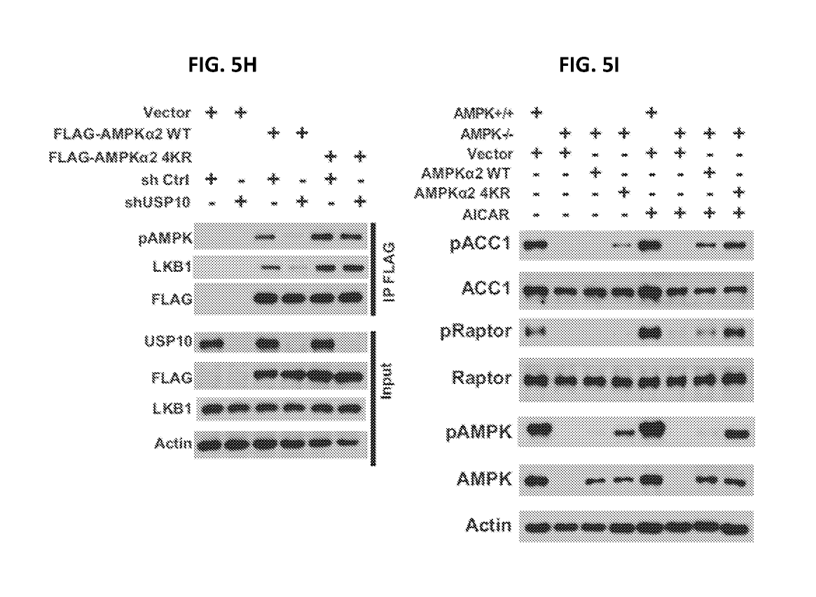

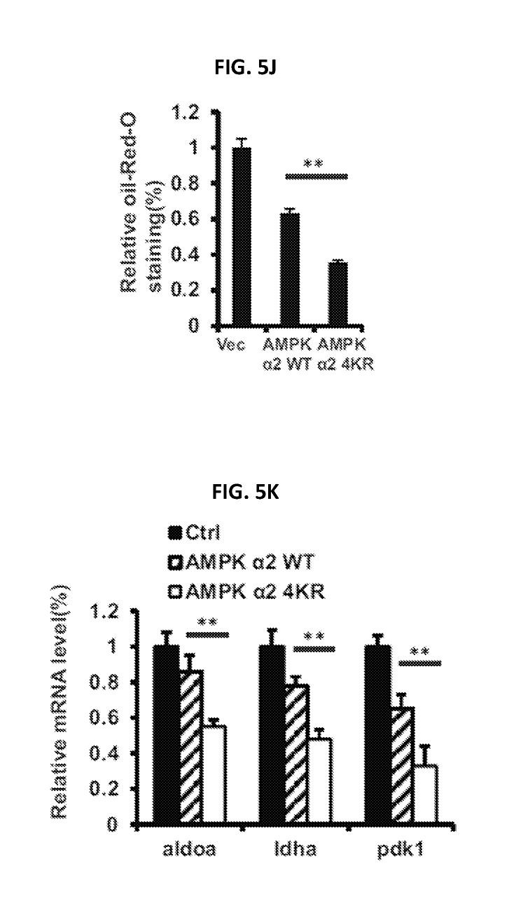

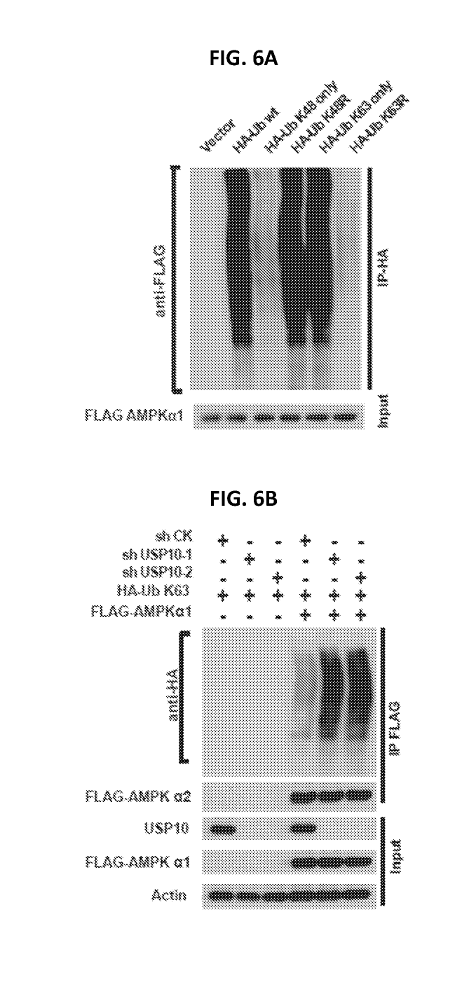

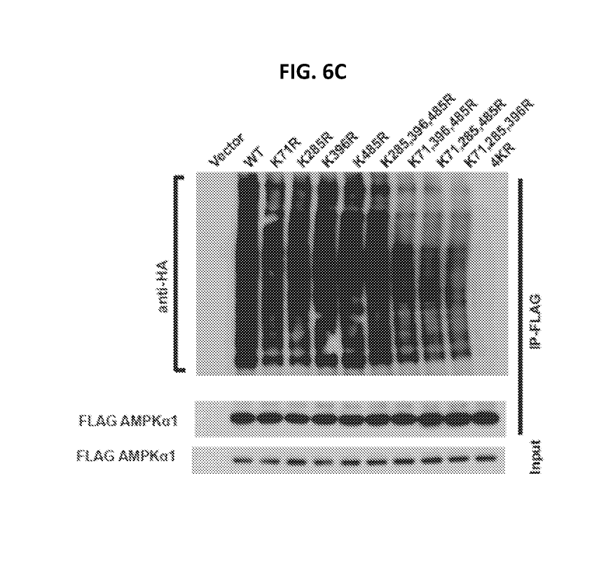

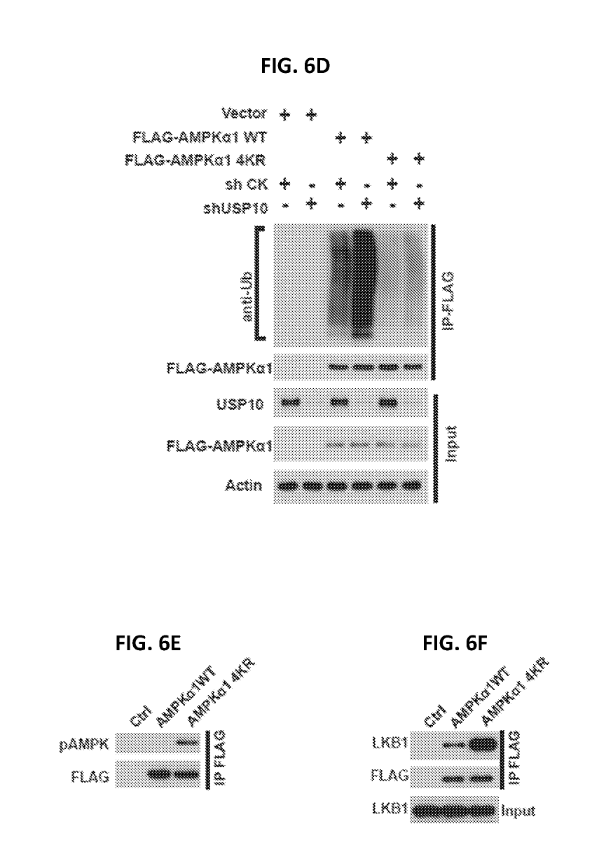

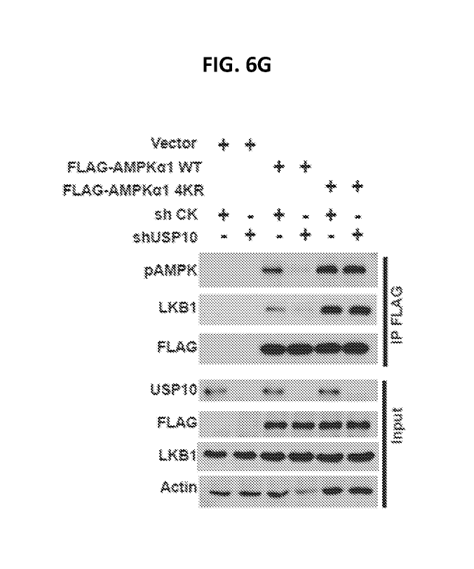

No change in AMPKα levels was observed when USP10 levels were modulated (FIGS. 1A and 2A). When the linkage of AMPKα ubiquitination was examined after USP10 knockdown, it was observed that AMPKα was mainly ubiquitinated through K63-specific chains (FIGS. 5A and 6A), and that these K63 ubiquitin chains were regulated by USP10 (FIGS. 5B and 6B). These results suggested that USP10 regulates the K63 ubiquitin chain on AMPKα.

The ubiquitination sites of AMPKα regulated by USP10 were further mapped. Analysis of a public proteomic database (Hornbeck et al., Nucleic Acids Res 43:D512-D520, 2015) suggested that AMPKα1 and α2 might be ubiquitinated at four potential sites (K71, K285, K396, and K485 in AMPKα1, and K60, K379, K391, and K470 in AMPKα2) (FIG. 5C). Mutants of the potential AMPKα ubiquitination sites (KR) were generated and tested to determine whether USP10 could regulate ubiquitination of these residues. For AMPKα2, single site mutants (K60R, K379R, K391R, and K470R) had little effect on total ubiquitination, while the triple K to R mutants had decreased ubiquitination levels and the quadruple K to R mutant (4KR) nearly abolished ubiquitination of AMPKα2 (FIG. 5D). These results indicated that these four lysine residues are the major ubiquitin sites on AMPKα2. For AMPKα1, one single site mutant (K71R) showed a significant decrease in ubiquitination, while the other single site mutants (K285R, K396R, and K485R) had little effect on ubiquitination (FIG. 6C). However, all three K to R mutants still showed stronger ubiquitination than the 4KR mutant (FIG. 6C), suggesting that all four sites are ubiquitinated and that K71 is more extensively ubiquitinated than the other three.

Studies were then conducted to test whether ubiquitination at these sites is regulated by USP10. As shown in FIGS. 5E and 6D, knockdown of USP10 resulted in increased ubiquitination of WT AMPKα. On the other hand, knockdown of USP10 did not affect ubiquitination levels of the AMPKα 4KR mutant (FIGS. 5E and 6D). These results suggested that these sites are major ubiquitination sites regulated by USP10.

To investigate the functional significance of AMPKα ubiquitination, activation of WT AMPK and the 4KR AMPK mutant was evaluated by determining Thr172 phosphorylation. Interestingly, the level of Thr172 phosphorylation for the 4KR mutant was much higher than that of WT AMPKα (FIGS. 5F and 6E). These results, together with the results showing that USP10 positively regulates AMPK activation, suggested that ubiquitination of AMPK negatively regulates its activation, and that USP10 removes the inhibitory ubiquitination of AMPK.

Experiments were then conducted to investigate how USP10-mediated ubiquitination affects its activation. LKB1 is the major kinase responsible for the phosphorylation of Thr172 of AMPKα under energy stress (Hawley et al. 2003, supra). As ubiquitination of AMPKα affects its phosphorylation, the interaction between LKB1 and WT AMPKα or the 4KR mutant was examined. As shown in FIGS. 5G and 6F, the AMPKα KR mutants bound much more strongly than WT to LKB1, consistent with higher AMPKα phosphorylation of these mutants (FIGS. 5F and 6E). These results suggested that ubiquitination of AMPKα interferes with its interaction with LKB1.

Further studies examined whether USP10 regulates AMPK activation by deubiquitinating AMPKα. As shown in FIGS. 5H and 6G, knockdown of USP10 decreased the phosphorylation of WT AMPKα, and disrupted LKB1-AMPK binding. However, knockdown of USP10 had no significant effect on phosphorylation of the AMPKα 4KR mutants, or on LKB1-AMPKα 4KR binding. These results suggested that ubiquitination of AMPKα blocks LKB1 binding and subsequent AMPK activation, and that USP10 regulates AMPK activation through deubiquitinating AMPKα.

To study the physiological function of AMPKα ubiquitination, AMPKα1α2^{-/-} MEF were reconstituted with WT AMPK α2 or the 4KR mutant, and the activation of AMPK was evaluated by assessing phosphorylation of AMPK and its substrates, ACC1 and Raptor. While WT AMPKα2 could partially restore the levels of phospho-AMPKα, phospho-ACC1, and phospho-Raptor, the AMPKα 4KR mutant was much more effective (FIG. 5I). Further, when lipogenesis was evaluated in these MEF cells, it was found that the AMPKα2 4KR mutant inhibited lipid droplet accumulation better than WT (FIG. 5J). The effect of expressing WT and the 4KR mutant on glycolytic gene expression also was investigated. The AMPKα2 4KR mutant had a much stronger effect toward inhibiting glycolytic gene expression (FIG. 5K). These results suggested that ubiquitination of AMPKα inhibits its activation and metabolic functions.

### Example 5

### AMPK Phosphorylates USP10 Under Energy Stress

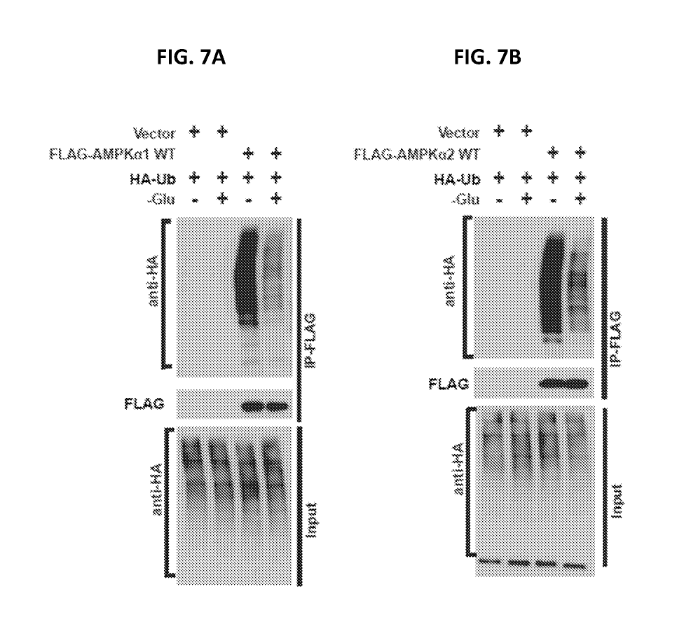

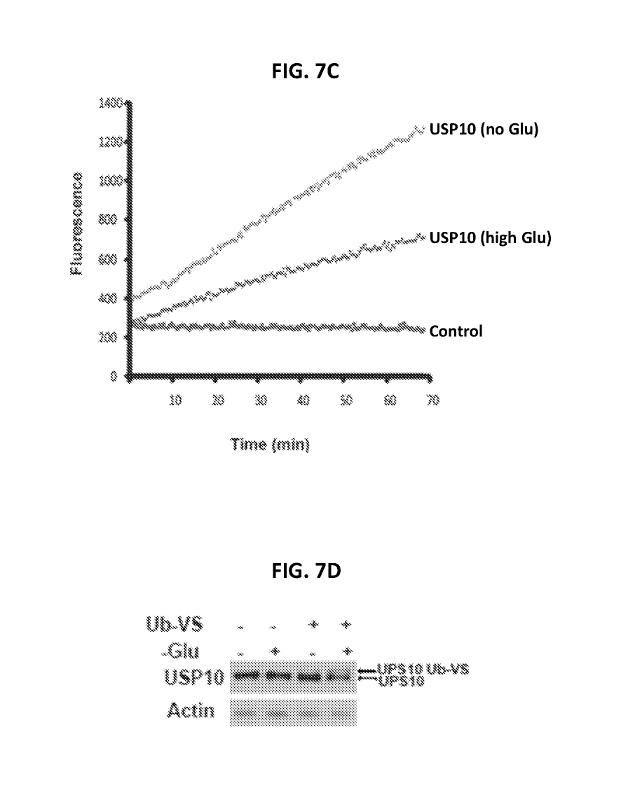

AMPK was activated under energy stress, accompanied by a decrease in AMPKα ubiquitination (FIGS. 7A and 7B). Since USP10 regulates AMPKα ubiquitination and activation, experiments were done to test whether USP10 activity increases during this process. As shown in FIG. 7C, the deubiquitinase activity of USP10 toward Ub-AMC increased significantly after glucose starvation treatment. To further confirm this, a modified deubiquitinating enzyme substrate, ubiquitin vinyl sulfone (Ub-VS), was used (Borodovsky et al., EMBO J20:5187-5196, 2001). Upon catalysis of Ub-VS, a covalent bond is formed between the substrate and the active cysteine residue of the USP10 enzyme, leading to a mobility shift of 8 kDa for the active enzyme in SDS-PAGE. After glucose starvation, a more apparent mobility shift of USP10 was observed (FIG. 7D), indicating increased USP10 activity after glucose starvation.

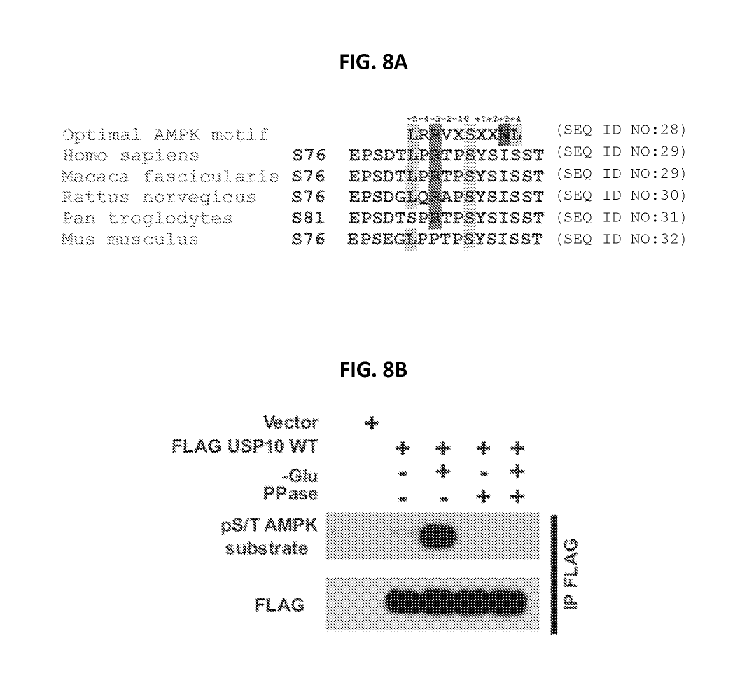

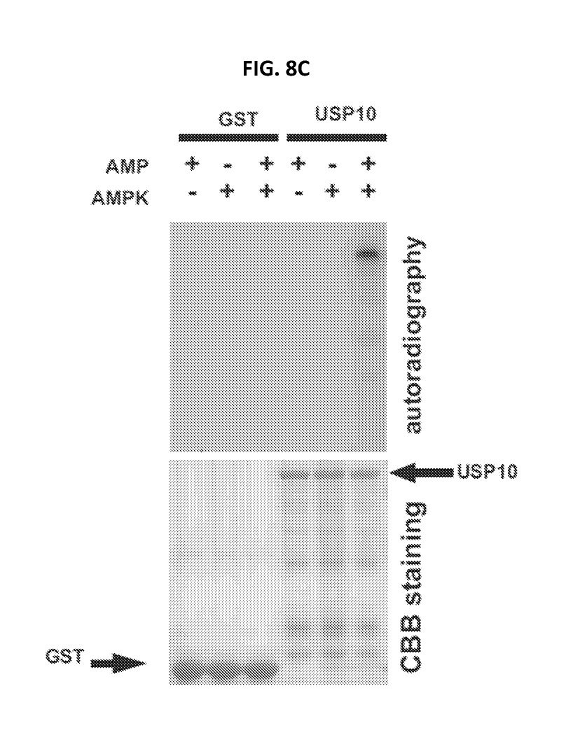

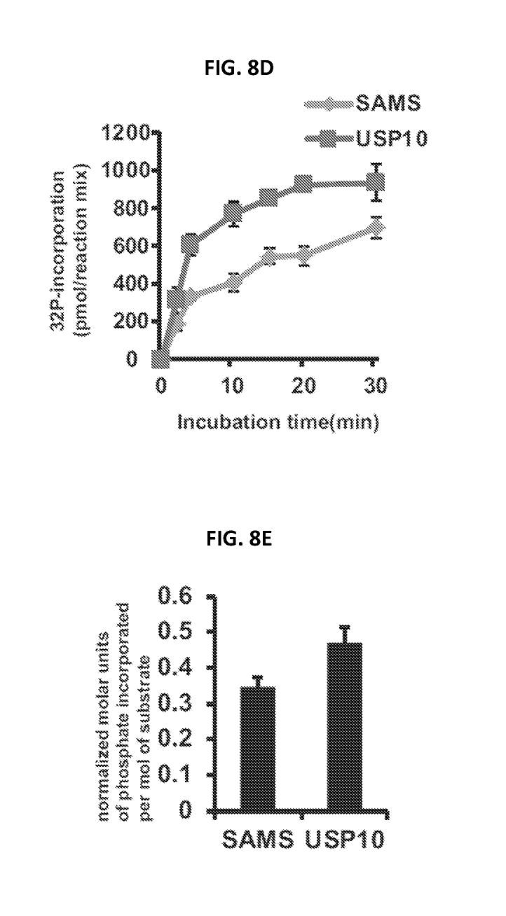

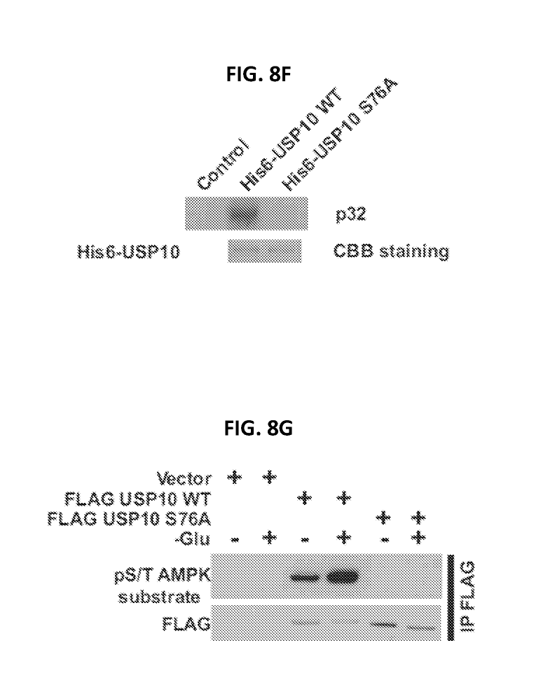

Studies were then conducted to explore how USP10 is regulated under energy stress. By sequence analysis, an optimal AMPK substrate motif was observed around Ser76 in the N-terminal portion of USP10 (FIG. 8A). Publically available mass spectrum databases also have indicated that Ser76 (S76) is phosphorylated in cells (Hornbeck et al., supra). To test whether USP10 could be phosphorylated by AMPK and whether such phosphorylation would affect USP10 activity, studies were carried out using a pan-AMPK substrate antibody. As shown in FIG. 8B, the phosphorylation of USP10 was increased significantly after glucose starvation, suggesting that USP10 might be a substrate of AMPK. To confirm this result, an in vitro kinase assay was performed, revealing that AMPK specifically phosphorylated USP10 but not GST in vitro (FIG. 8C), and further suggesting that USP10 is an AMPK substrate. To further confirm phosphorylation of USP10 by AMPK, the phosphorylation of a synthesized USP10 S76 peptide was compared with the phosphorylation of a SAMS peptide (a known AMPK substrate) in an in vitro kinase assay (Davies et al., Eur J Biochem 186:123-128, 1989). The initial rate of phosphorylation for the USP10 peptide was 155±36 pmol/min/reaction mix, which was higher than that of SAMS (95±18 pmol/min/reaction mix) (FIG. 8D). The maximal stoichiometry of phosphorylation of the USP10 peptide by AMPK was 0.46±0.04 mol/mol, which also was higher than that of SAMS (0.35±0.02 mol/mol) (FIG. 8E). These results indicated that USP10 is an in vitro substrate of AMPK.

Further experiments tested whether the serine at position 76 (S76) of USP10 is the phosphorylation site. After mutation of S76 to Ala, phosphorylation of USP10 by AMPK in vitro was totally abolished (FIG. 8F). Further, the S76A mutant totally abolished the phosphorylation signal of USP10 in cells following glucose starvation (FIG. 8G). These results indicated that AMPK phosphorylates USP10 at Ser76.

### Example 6

### Phosphorylation of USP10 by AMPK Increases its Activity

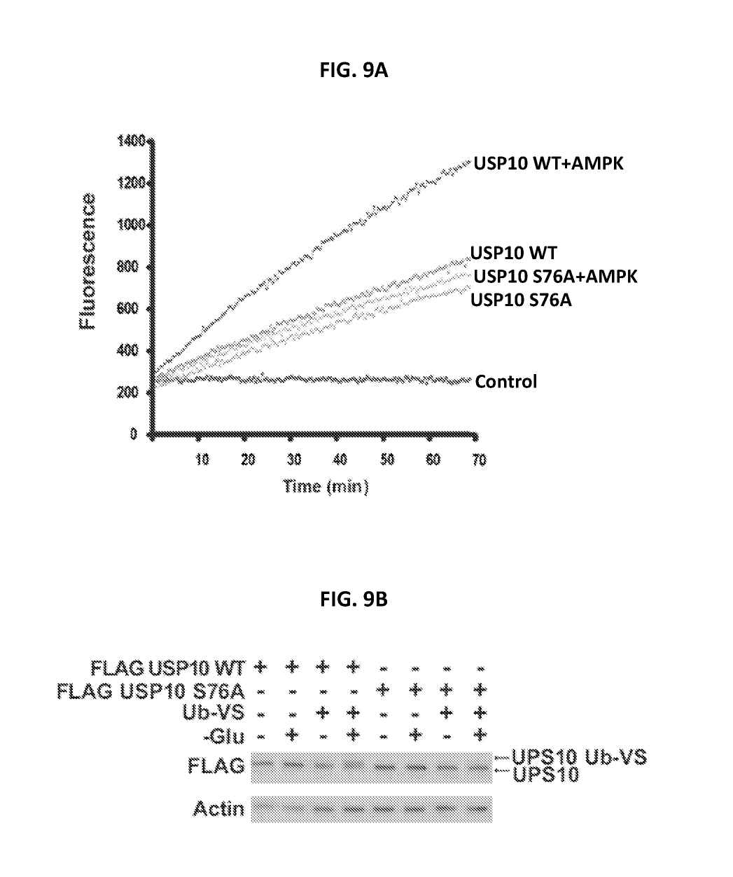

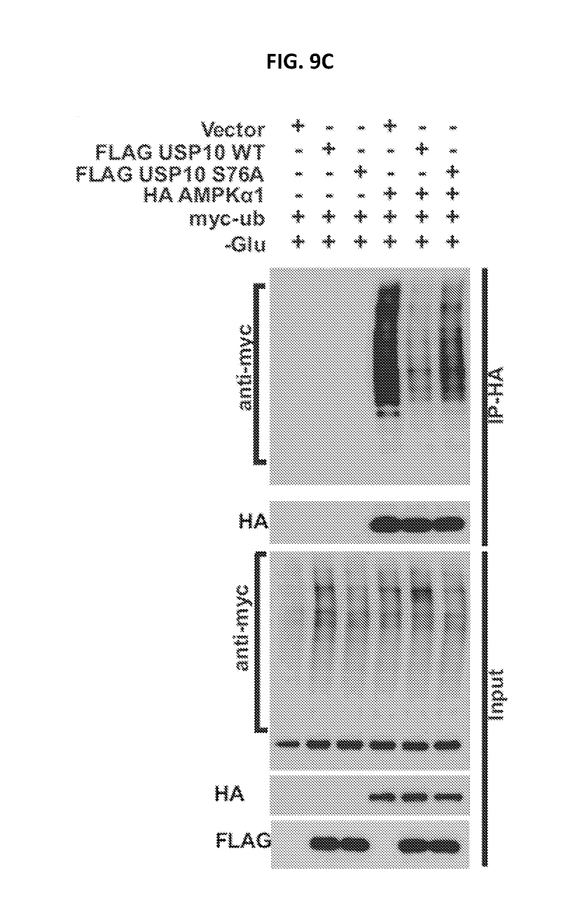

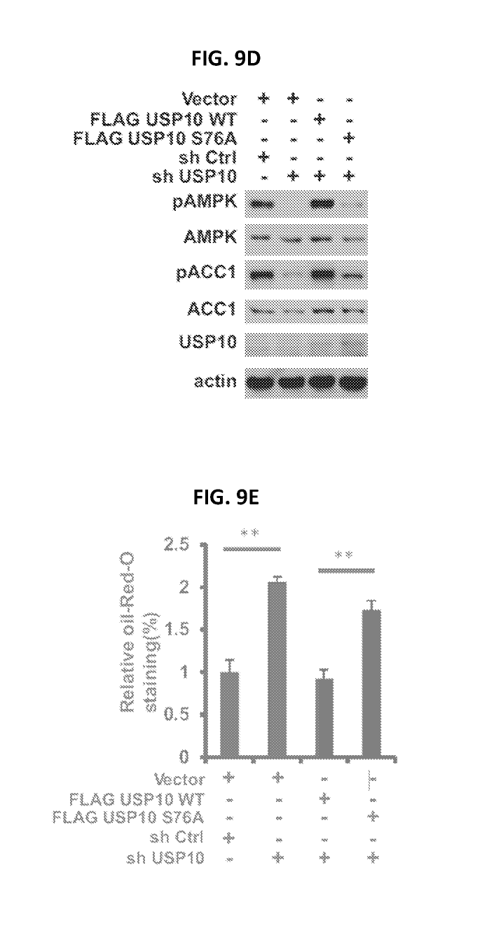

Because USP10 deubiquitinase activity and phosphorylation at S76 were increased following glucose starvation, studies were conducted to test whether S76 phosphorylation affects USP10 activity. USP10 was phosphorylated using active AMPK in vitro, and its activity toward Ub-AMC was evaluated. Compared to unphosphorylated USP10, phosphorylated USP10 showed much higher activity toward Ub-AMC (FIG. 9A). However, the activity of the S76A mutant remained low even after incubating with active AMPK (FIG. 9A). The activity of WT USP10 and the S76A mutant also was compared in cells using the Ub-VS labeling assay. While the activity of WT USP10 increased significantly after glucose starvation, the activity of the S76A mutant did not increase (FIG. 9B). These results suggested that S76 phosphorylation of USP10 increases its deubiquitinase activity. Further tests were conducted to determine whether phosphorylation of S76 on USP10 would affect the ubiquitination and activation of AMPKα in cells. WT USP10 and the S76A mutant were reconstituted in USP10 knockdown cells, and AMPKα ubiquitination was evaluated. As shown in FIG. 9C, AMPKα ubiquitination was stronger in cells expressing the S76A mutant, suggesting that the S76A mutant has much less deubiquitinase activity toward AMPKα than WT USP10, and indicating that phosphorylated S76 is important for the deubiquitinase activity of USP10 toward AMPKα.

To study the physiological significance of S76 phosphorylation, WT USP10 or the S76A mutant was again reconstituted in USP10 knockdown cells, and phosphorylation was measured. In cells reconstituted with USP10 S76A, the phosphorylation of AMPK and ACC1 was much lower than in cells reconstituted with WT USP10 (FIG. 9D), indicating that the phosphorylation of USP10 is important for AMPK activation. The effect of USP10 phosphorylation on lipogenesis and glycolytic gene expression also was assessed. While reconstitution of WT USP10 reversed misregulation of lipid droplet formation and glycolytic gene expression caused by USP10 deficiency, expression of the S76A mutant only had a partial rescue effect (FIGS. 9E and 9F). These results suggested that the phosphorylation of USP10 is important for its function in AMPK activation and cellular metabolism. Taken together, these results indicated that AMPK phosphorylates USP10 at S76, which activates USP10 and facilitates further activation of AMPK under energy stress, forming a feedforward loop.

### Example 7

### Depletion of hepatic USP10 Leads to Multiple Metabolic Defects

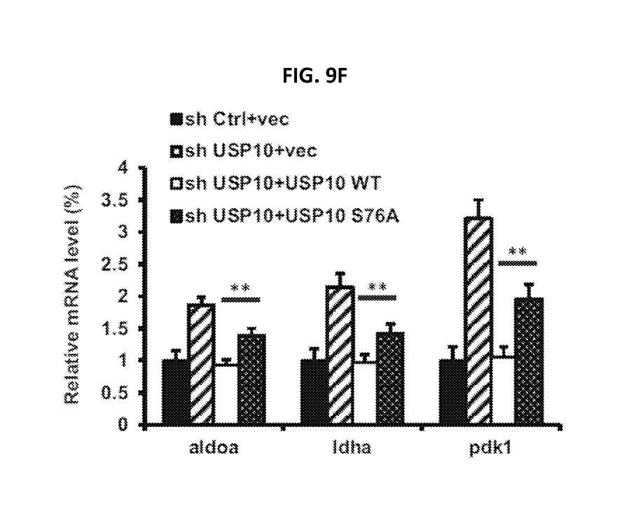

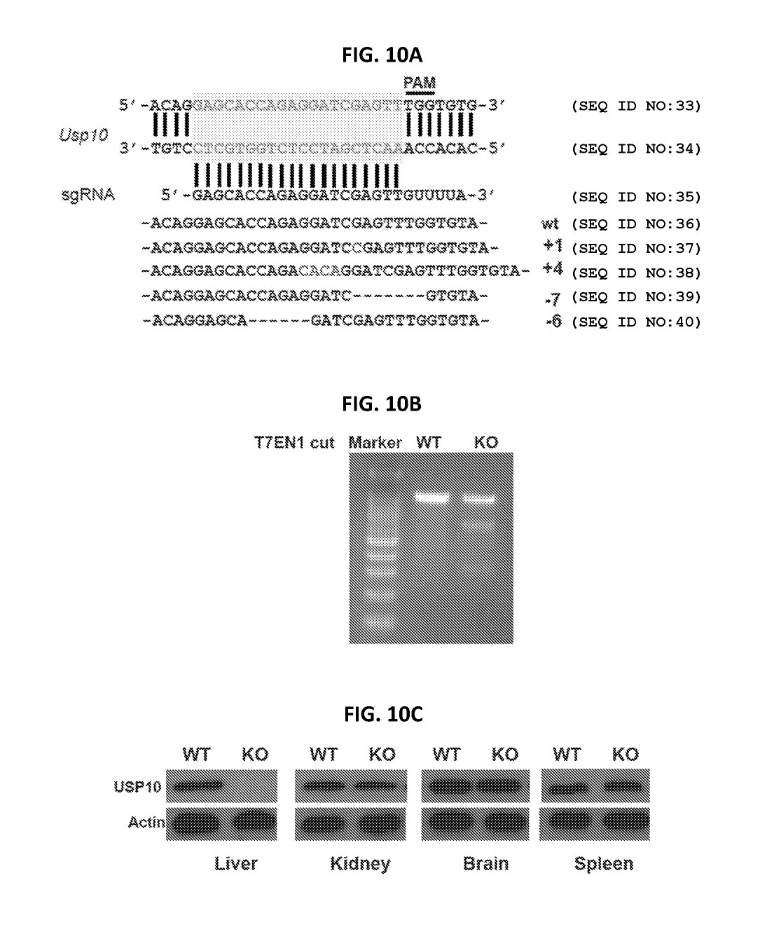

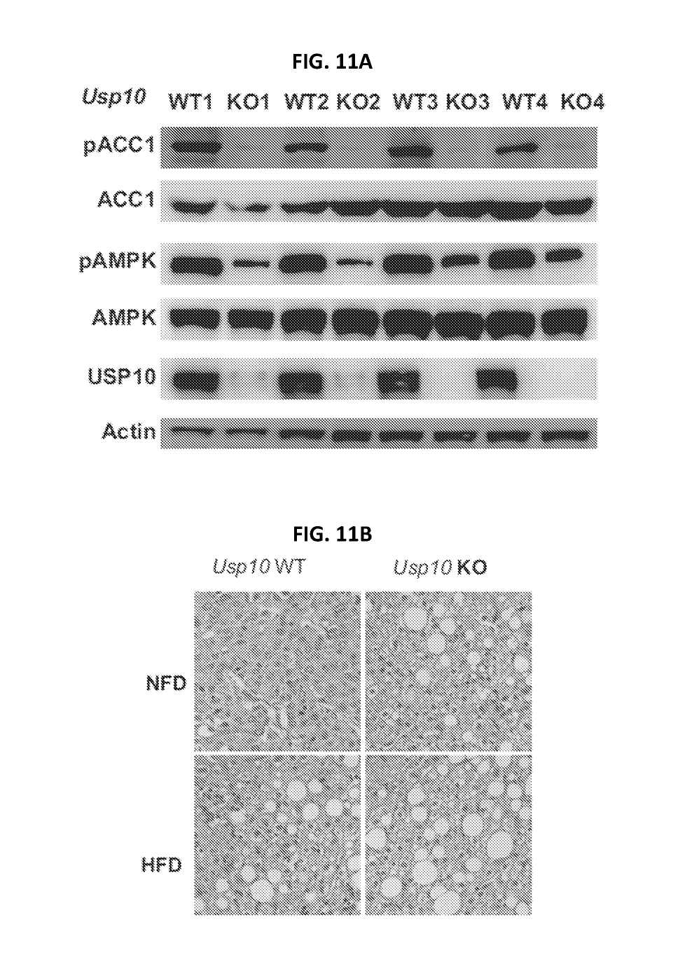

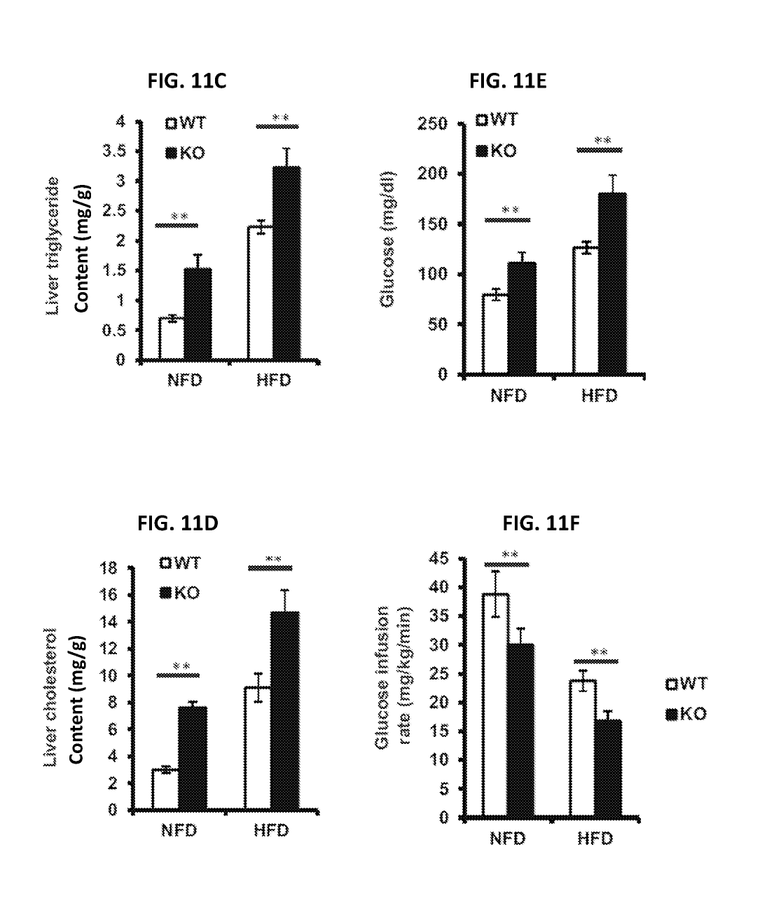

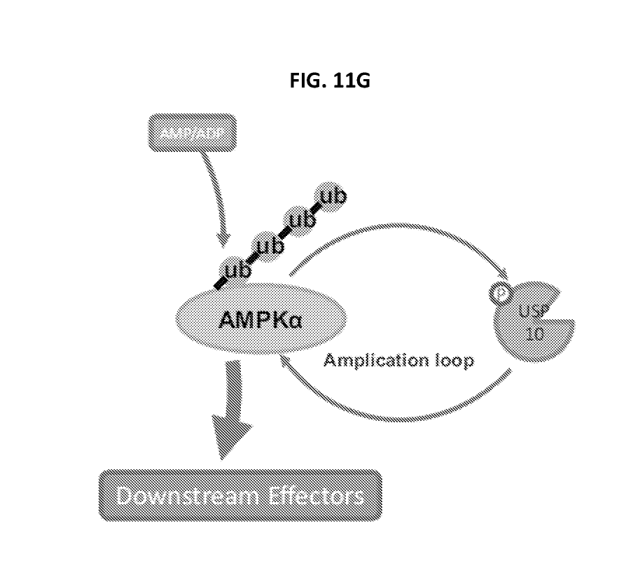

The physiological function of USP10 in animal models was evaluated using an adenovirus-based gene targeting approach to specifically knock out Usp10 in the mouse liver (FIGS. 10A and 10B). Because of the high tropism of adenovirus for hepatocytes, a total loss of USP10 protein was observed in the livers of adenovirus-injected mice, with no sign of USP10 loss in kidney, brain, or spleen tissue as judged by immunoblotting of lysates from these tissues (FIG. 10C). Consistent with the cell based assays, deletion of the Usp10 gene in the liver resulted in a drastic decrease of phospho-AMPK, as well as phospho-ACC1 (FIG. 11A), indicating that AMPK is not properly activated in the absence of USP10 in vivo. Further, Usp10 liver-specific knockout mice showed multiple metabolic defects. For example, knockout of Usp10 significantly increased hepatic triglyceride and cholesterol contents, as measured by a colorimetric assay and HE staining under both normal fat diet and high fat diet conditions (FIGS. 9B, 9C, and 9D). Usp10 knockout mice also showed increased blood glucose levels and decreased glucose infusion rates (FIGS. 1E and 1F), indicating that USP10 regulates hepatic glucose metabolism. These phenotypes are consistent with the function of AMPK in gluconeogenesis and lipogenesis (Assifi et al., supra; Foretz et al., Diabetes 54:1331-1339, 2005; Steinberg and Kemp, supra). Taken together, these results suggested that USP10 plays a key role in AMPK activation and controls lipid and glucose metabolism in vivo.

Protein kinases are known to utilize downstream signaling members to remove inhibitory effects on upstream members, thus increasing the total flux through the pathway (Avraham and Yarden, Nat Rev Mol Cell Bio 12:104-117, 2011; Pomerening, Febs Lett 583:3388-3396, 2009). In the model presented herein (FIG. 11G), AMPK is initially activated through AMP or ADP binding and phosphorylation of a threonine residue (Thr-172) within the activation loop of its kinase domain. After AMPK is partially activated, AMPK activates USP10 to remove the inhibitory ubiquitination on itself, forming a positive feedforward loop. In this way, the initial minor activation of AMPK is amplified into a sustained response. Disruption of this feedforward loop would lead to improper AMPK activation under energy stress. Thus this feedforward activation mechanism ensures precise regulation of AMPK activity.

Since USP10 also regulates p53 expression and suppresses tumor cell growth (Yuan et al., supra), phosphorylation of USP10 under energy stress might contribute to p53 level increase and tumor suppression. AMPK also has been shown to activate p53 (Jones et al., Mol Cell 18:283-293, 2005). Thus, the feedforward loop of AMPK-USP10 also may activate p53 through AMPK.

AMPK plays important roles in a number of cell signaling pathways, including cell growth, autophagy, and metabolism (Hardie, Genes Dev 25:1895-1908, 2011; Mihaylova and Shaw, supra). The key role of AMPK places it as an ideal therapeutic target for the treatment of obesity, insulin resistance, type 2 diabetes, metabolic syndromes, neurological disorders, and cancer (Steinberg and Kemp, supra). In light of the data described herein, which demonstrate an important role for USP10 in AMPK activation, it appears that USP10 could be a potential target for these diseases.

### Example 8

### USP10 Interacts with and Regulates mTOR Signaling

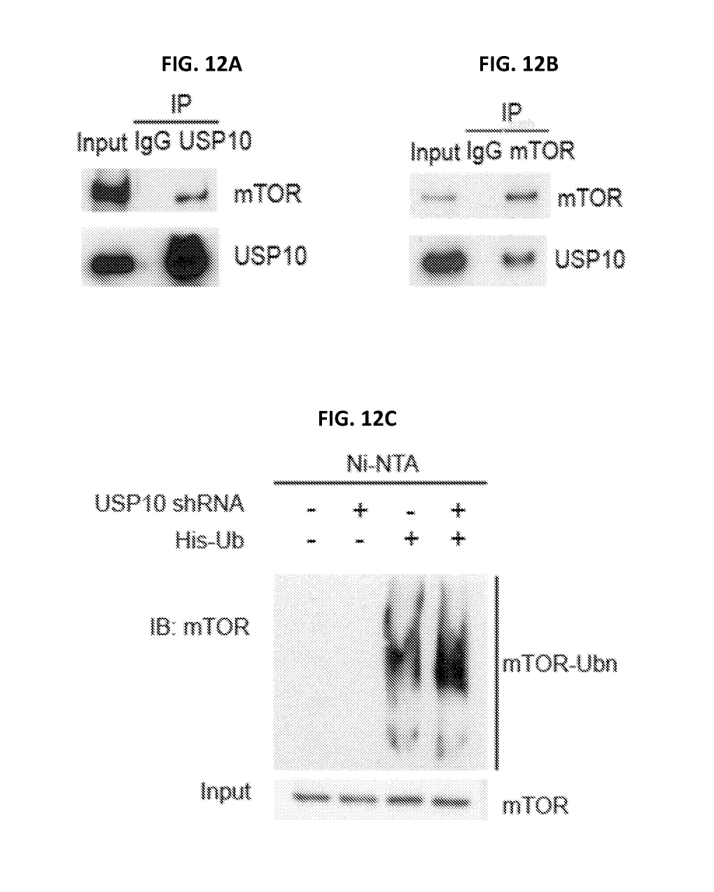

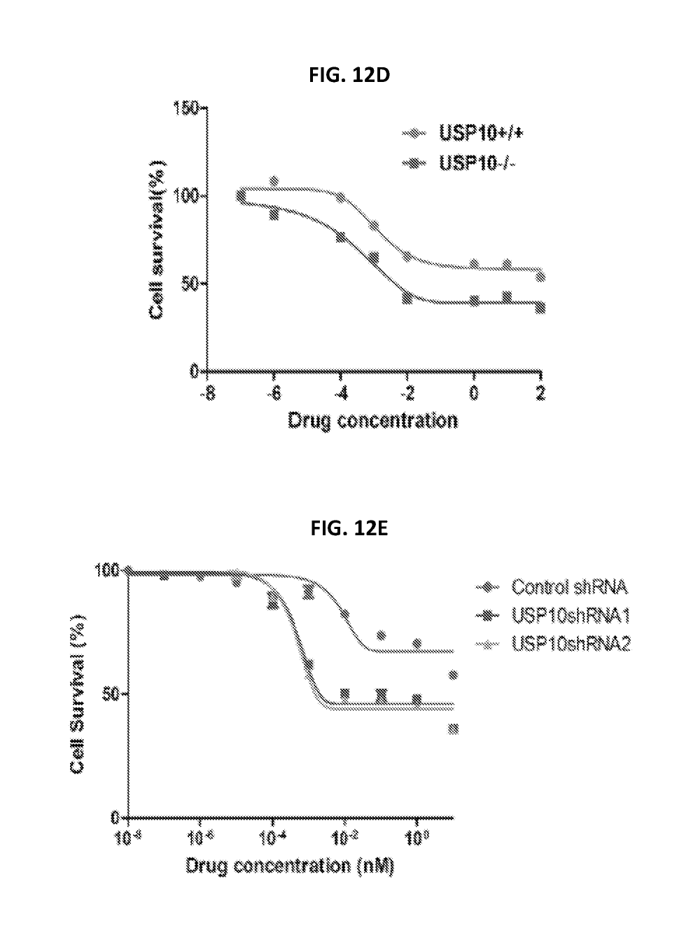

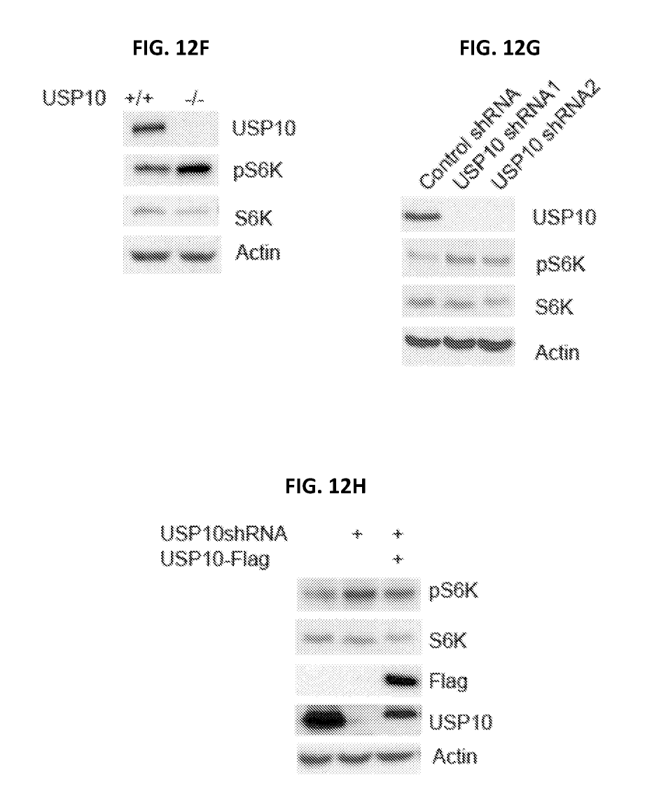

mTOR is a serine/threonine protein kinase that regulates cell growth, cell proliferation, cell motility, cell survival, protein synthesis, autophagy, and transcription (Hay and Sonenberg, supra), and is an important tumor target. Experiments were conducted to assess whether USP10 might interact with and/or affect the activity of mTOR. First, Co-IP assays were conducted, demonstrating that USP10 interacts with mTOR (FIGS. 12A and 12B). Further immunoblotting studies demonstrated that USP10 regulates mTOR ubiquitination (FIG. 12C), as ubiquitination of mTOR was increased when USP10 was knocked down with shRNA.

USP10 also was found to be involved in the response of cancer cells to everolimus, an mTOR inhibitor. As shown in FIG. 12D, survival of cancer cells treated with everolimus was decreased in the absence of USP10. Thus, the inhibitor had a greater effect on cancer cells in the absence of USP10, demonstrating that USP10 regulates the response of cancer cells to this mTOR inhibitor. Studies using shRNA knockdown of USP10 showed similar results, as the mTOR inhibitor had a greater effect on survival of USP10 knockdown cells than on cells treated with a control shRNA (FIG. 12E).

To determine the effect of USP10 on mTOR signaling, experiments were conducted to assess phosphorylation of the mTOR substrate, S6K, in the presence and absence of USP10. These studies demonstrated that USP10 negatively regulates mTOR signaling. USP10 knockout (FIG. 12F) or knockdown (FIG. 12G) resulted in increased levels of phospho-S6K, while reconstitution of USP10 in knockdown cells resulted in decreased levels of phospho-S6K (FIG. 12H). Thus, deubiquitination of mTOR by USP10 reduces the activity of mTOR, while increased mTOR ubiquitination in USP10 knockout or knockdown cells is associated with increased mTOR kinase activity.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for increasing the activity of AMP-activated protein kinase (AMPK) in a cell, comprising contacting the cell with an agent effective to increase the level or deubiquitinase activity of USP10 in the cell.

2. The method of claim 1, wherein the agent is a USP10 polypeptide.

3. The method of claim 2, wherein the USP10 polypeptide comprises the amino acid sequence set forth in SEQ ID NO:2.

4-5. (canceled)

6. The method of claim 2, wherein the USP10 polypeptide is a biologically active fragment of the USP10 polypeptide having the sequence set forth in SEQ ID NO:2.

7. (canceled)

8. The method of claim 1, wherein the agent is a nucleic acid encoding a USP10 polypeptide.

9. The method of claim 8, wherein the nucleic acid comprises the sequence set forth in nucleotides 143-2536 of SEQ ID NO:1.

10-11. (canceled)

12. The method of claim 8, wherein the nucleic acid encodes a biologically active fragment of a USP10 polypeptide having the sequence set forth in SEQ ID NO:2.

13-14. (canceled)

15. The method of claim 1, wherein the cell is in a subject identified as having a clinical condition selected from the group consisting of obesity, type 2 diabetes, insulin resistance, or metabolic syndrome.

16. The method of claim 1, wherein the cell is in a subject identified as having cancer.

17-18. (canceled)

19. A method for reducing the activity of mammalian target of rapamycin (mTOR) in a cell, comprising contacting the cell with an agent effective to increase the level or deubiquitinase activity of USP10 in the cell.

20. The method of claim 19, wherein the agent is a USP10 polypeptide.

21. The method of claim 20, wherein the USP10 polypeptide comprises the amino acid sequence set forth in SEQ ID NO:2.

22-23. (canceled)

24. The method of claim 20, wherein the USP10 polypeptide is a biologically active fragment of the USP10 polypeptide having the sequence set forth in SEQ ID NO:2.

25. (canceled)

26. The method of claim 19, wherein the agent is a nucleic acid encoding a USP10 polypeptide.

27. The method of claim 26, wherein the nucleic acid comprises the sequence set forth in nucleotides 143-2536 of SEQ ID NO:1.

28-29. (canceled)

30. The method of claim 26, wherein the nucleic acid encodes a biologically active fragment of a USP10 polypeptide having the sequence set forth in SEQ ID NO:2.

31-32. (canceled)

33. The method of claim 19, wherein the cell is in a subject identified as having cancer.

34. (canceled)

35. The method of claim 33, wherein the subject is a human.

36-41. (canceled)

42. A method for increasing the activity of AMPK in a cell, comprising contacting the cell with an agent effective to increase, in the cell, the level of an AMPK polypeptide that lacks one or more ubiquitination sites.

43. The method of claim 42, wherein the agent is the AMPK polypeptide that lacks one or more ubiquitination sites.

44. The method of claim 42, wherein the agent is a nucleic acid encoding the AMPK polypeptide that lacks one or more ubiquitination sites.

45. The method of claim 43 or claim 44, wherein the AMPK polypeptide is an AMPKα1 polypeptide comprising a substitution of one or more of the lysine at position 71, the lysine at position 285, the lysine at position 396, and the lysine at position 485.

46-48. (canceled)

49. The method of claim 43 or claim 44, wherein the AMPK polypeptide is an AMPKα2 polypeptide comprising a substitution of one or more of the lysine at position 60, the lysine at position 379, the lysine at position 391, and the lysine at position 470.

50-51. (canceled)


SEQUENCE LISTING (42 sequences)

SEQ ID NO: 1 - DNA, 3399 bp, Homo sapiens
ctccccgcgccccgcggcgcgcggccagtgcgcaggcgcggcggccgatgcgagtgtgtatgtgcgggcgagaagatggcggcggcgggggaagcagcgtgagcagccggaggatcgcggagtcccaatgaaacgggcagccatggccctccacagcccgcagtatatttttggagattttagccctgatgaattcaatcaattctttgtgactcctcgatcttcagttgagcttcctccatacagtggaacagttctgtgtggcacacaggctgtggataaactacctgatggacaagaatatcagagaattgagtttggtgtcgatgaagtcattgaacccagtgacactttgccgagaacccccagctacagtatttcaagcacactgaaccctcaggcccctgaatttattctcggttgtacagcttccaaaataacccctgatggtatcactaaagaagcaagctatggctccatcgactgccagtacccaggctctgccctcgctttggatggaagttctaatgtggaggcggaagttttggaaaatgatggtgtctcaggtggtcttggacaaagggagcgtaaaaagaagaaaaagcggccacctggatattacagctatttgaaagatggtggcgatgatagtatctccacagaagccctggtcaatggccatgccaattcagcagtcccgaacagtgtcagtgcagaggatgcagaatttatgggtgacatgcccccgtcagttacgcccaggacttgtaacagcccccagaactccacagactctgtcagtgacattgtgcctgacagtcctttccccggagcactcggcagtgacaccaggactgcagggcagccagaggggggccccggggctgattttggtcagtcctgcttccctgcagaggctggcagagacaccctgtcaaggacagctggggctcagccctgcgttggtaccgatactactgaaaaccttggagttgctaatggacaaatacttgaatcctcgggtgagggcacagctaccaacggggtggagttgcacaccacggaaagcatagacttggacccaaccaaacccgagagtgcatcacctcctgctgacggcacgggctctgcatcaggcacccttcctgtcagccagcccaagtcctgggccagcctctttcatgattctaagccctcttcctcctcgccggtggcctatgtggaaactaagtattcccctcccgccatatctcccctggtttctgaaaagcaggttgaagtcaaagaagggcttgttccggtttcagaggatcctgtagccataaagattgcagagttgctggagaatgtaaccctaatccataaaccagtgtcgttgcaaccccgtgggctgatcaataaagggaactggtgctacattaatgctacactgcaggcattggttgcttgcccgccgatgtaccacctgatgaagttcattcctctgtattccaaagtgcaaaggccttgtacgtcaacacccatgatagacagctttgttcggctaatgaatgagttcactaatatgccagtacctccaaaaccccgacaagctcttggagataaaatcgtgagggatattcgccctggagctgcctttgagcccacatatatttacagactcctgacagttaacaagtcaagcctgtctgaaaagggtcgacaagaagatgctgaggaatacttaggcttcattctaaatggacttcatgaggaaatgttgaacctaaagaagcttctctcaccaagtaatgaaaaacttacgatttccaacggccccaaaaaccactcggtcaatgaagaagagcaggaagaacaaggtgaaggaagcgaggatgaatgggaacaagtgggcccccggaacaagacttccgtcacccgccaggcggattttgttcagactccaatcaccggcatttttggtggacacatcaggtctgtggtttaccagcagagttcaaaagaatctgccactttgcagccatttttcacgttgcagttggatatccagtcagacaagatacgcacagtccaggatgcactggagagcttggtggcaagagaatctgtccaaggttataccacaaaaaccaaacaagaggttgagataagtcgaagagtgactctggaaaaactccctcctgtcctcgtgctgcacctgaaacgattcgtttatgagaagactggtgggtgccagaagcttatcaaaaatattgaatatcctgtggacttggaaattagtaaagaactgctttctccaggggttaaaaataagaattttaaatgccaccgaacctatcggctctttgcagtggtctaccatcacggcaacagtgcgacgggcggccattacactacagacgtcttccagatcggtctgaatggctggctgcgcatcgatgaccagacagtcaaggtgatcaaccagtaccaggtggtgaaaccaactgctgaacgcacagcctacctcctgtattaccgccgagtggacctgctgtaaaccctgtgtgcgctgtgtgtgcgcccagtgcccgcttcgtaggacaccacctcacactcacttcccgcctctctttagtggctctttagagagaaactctttctccctttgcaaaaatgggctagaatgaaaaggagatgccttggggttcgtgcacaacacagcttctgttgactctaacttccaaatcaaaatcatttggttgaaacagactgttgcttgattttagaaaatacacaaaaacccatatttctgaaataatgctgattcctgagataagaaagtggatttgatccccagtctcattgcttagtagaataaatcctgcaccagcaacaacacttgtaaatttgtgaaaatgaattttatctttccttaaaaaagaaattttttaatccatcacacttttcttccctaccctttagtttttgataaatgataaaaatgagccagttatcaaagaagaactagttcttacttcaaaagaaaaataaacataaaaaataagttgctggttcctaacaggaaaaattttaataattgtactgagagaaactgcttacgtacacattgcagatcaaatatttggagttaaaatgttagtctacatagatgggtgattgtaactttattgccattaaaagatttcaaattgcattcatgcttctgtgtacacataatgaaaaatgggcaaataatgaagatctctccttcagtctgctctgtttaattctgctgtctgctcttctctaatgctgcgtccctaattgtacacagtttagtgatatctaggagtataaagttgtcgcccatcaataaaaatcacaaagttggtttaaaaaaaaaaaaaaaaaaaaaaa

SEQ ID NO: 2 - protein, 798 aa, Homo sapiens
MALHSPQYIFGDFSPDEFNQFFVTPRSSVELPPYSGTVLCGTQAVDKLPDGQEYQRIEFGVDEVIEPSDTLPRTPSYSISSTLNPQAPEFILGCTASKITPDGITKEASYGSIDCQYPGSALALDGSSNVEAEVLENDGVSGGLGQRERKKKKKRPPGYYSYLKDGGDDSISTEALVNGHANSAVPNSVSAEDAEFMGDMPPSVTPRTCNSPQNSTDSVSDIVPDSPFPGALGSDTRTAGQPEGGPGADFGQSCFPAEAGRDTLSRTAGAQPCVGTDTTENLGVANGQILESSGEGTATNGVELHTTESIDLDPTKPESASPPADGTGSASGTLPVSQPKSWASLFHDSKPSSSSPVAYVETKYSPPAISPLVSEKQVEVKEGLVPVSEDPVAIKIAELLENVTLIHKPVSLQPRGLINKGNWCYINATLQALVACPPMYHLMKFIPLYSKVQRPCTSTPMIDSFVRLMNEFTNMPVPPKPRQALGDKIVRDIRPGAAFEPTYIYRLLTVNKSSLSEKGRQEDAEEYLGFILNGLHEEMLNLKKLLSPSNEKLTISNGPKNHSVNEEEQEEQGEGSEDEWEQVGPRNKTSVTRQADFVQTPITGIFGGHIRSVVYQQSSKESATLQPFFTLQLDIQSDKIRTVQDALESLVARESVQGYTTKTKQEVEISRRVTLEKLPPVLVLHLKRFVYEKTGGCQKLIKNIEYPVDLEISKELLSPGVKNKNFKCHRTYRLFAVVYHHGNSATGGHYTTDVFQIGLNGWLRIDDQTVKVINQYQVVKPTAERTAYLLYYRRVDLL

SEQ ID NO: 3 - DNA, 24 bp, Artificial Sequence
  synthetic primer
cggttccgatgccctgaggctctt

SEQ ID NO: 4 - DNA, 24 bp, Artificial Sequence
  synthetic primer
cgtcacacttcatgatggaattga

SEQ ID NO: 5 - DNA, 20 bp, Artificial Sequence
  synthetic primer
gtgggaagaaggagaacctg

SEQ ID NO: 6 - DNA, 20 bp, Artificial Sequence
  synthetic primer
ctggagtgttgatggagcag

SEQ ID NO: 7 - DNA, 22 bp, Artificial Sequence
  synthetic primer
tgtctccagcaaagactactgt

SEQ ID NO: 8 - DNA, 23 bp, Artificial Sequence
  synthetic primer
gactgtacttgacaatgttggga

SEQ ID NO: 9 - DNA, 24 bp, Artificial Sequence
  synthetic primer
acaaggagagcttcggggtggatc

SEQ ID NO: 10 - DNA, 24 bp, Artificial Sequence
  synthetic primer
ccacgtcgcagtttggatttatgc

SEQ ID NO: 11 - DNA, 17 bp, Artificial Sequence
  synthetic primer
aggcaccagggcgtgat

SEQ ID NO: 12 - DNA, 24 bp, Artificial Sequence
  synthetic primer
cgtcacacttcatgatggaattga

SEQ ID NO: 13 - DNA, 19 bp, Artificial Sequence
  synthetic primer
gcccgttatgccagtatct

SEQ ID NO: 14 - DNA, 20 bp, Artificial Sequence
  synthetic primer
agccaagaccttctctgtaa

SEQ ID NO: 15 - DNA, 18 bp, Artificial Sequence
  synthetic primer
ggttgagagtgcttatga

SEQ ID NO: 16 - DNA, 19 bp, Artificial Sequence
  synthetic primer
aacactaaggaagacatca

SEQ ID NO: 17 - DNA, 19 bp, Artificial Sequence
  synthetic primer
cggatcagaaaccgacaca

SEQ ID NO: 18 - DNA, 21 bp, Artificial Sequence
  synthetic primer
actgaacattctggctggtga

SEQ ID NO: 19 - protein, 13 aa, Homo sapiens
TLPRTPSYSISST

SEQ ID NO: 20 - protein, 21 aa, Homo sapiens
QKIRSLDVVGKIRREIQNLKL

SEQ ID NO: 21 - protein, 21 aa, Homo sapiens
EHEWFKQDLPKYLFPEDPSYS

SEQ ID NO: 22 - protein, 21 aa, Homo sapiens
RHTLDELNPQKSKHQGVRKAK

SEQ ID NO: 23 - protein, 21 aa, Homo sapiens
RSIDDEITEAKSGTATPQRSG

SEQ ID NO: 24 - protein, 21 aa, Homo sapiens
QKIRSLDVVGKIKREIQNLKL

SEQ ID NO: 25 - protein, 21 aa, Homo sapiens
RMPPLIADSPKARCPLDALNT

SEQ ID NO: 26 - protein, 21 aa, Homo sapiens
RCPLDALNTTKPKSLAVKKAK

SEQ ID NO: 27 - protein, 21 aa, Homo sapiens
VDNRSYLLDFKSIDDEVVEQR

SEQ ID NO: 28 - protein, 10 aa, Artificial Sequence
  optimized sequence
  misc_feature (5)..(5): Xaa can be any naturally occurring amino acid
  misc_feature (7)..(8): Xaa can be any naturally occurring amino acid
LRRVXSXXNL

SEQ ID NO: 29 - protein, 17 aa, Homo sapiens
EPSDTLPRTPSYSISST

SEQ ID NO: 30 - protein, 17 aa, Rattus norvegicus
EPSDGLQRAPSYSISST

SEQ ID NO: 31 - protein, 17 aa, Pan troglodytes
EPSDTSPRTPSYSISST

SEQ ID NO: 32 - protein, 17 aa, Mus musculus
EPSEGLPPTPSYSISST

SEQ ID NO: 33 - DNA, 31 bp, Mus musculus
acaggagcaccagaggatcgagtttggtgtg

SEQ ID NO: 34 - DNA, 31 bp, Mus musculus
cacaccaaactcgatcctctggtgctcctgt

SEQ ID NO: 35 - DNA, 26 bp, Artificial Sequence
  synthetic
gagcaccagaggatcgagttguuuua

SEQ ID NO: 36 - DNA, 31 bp, Mus musculus
acaggagcaccagaggatcgagtttggtgta

SEQ ID NO: 37 - DNA, 32 bp, Artificial Sequence
  synthetic
acaggagcaccagaggatccgagtttggtgta

SEQ ID NO: 38 - DNA, 35 bp, Artificial Sequence
  synthetic
acaggagcaccagacacaggatcgagtttggtgta

SEQ ID NO: 39 - DNA, 24 bp, Artificial Sequence
  synthetic
acaggagcaccagaggatcgtgta

SEQ ID NO: 40 - DNA, 25 bp, Artificial Sequence
  synthetic
acaggagcagatcgagtttggtgta

SEQ ID NO: 41 - protein, 559 aa, Homo sapiens
MRRLSSWRKMATAEKQKHDGRVKIGHYILGDTLGVGTFGKVKVGKHELTGHKVAVKILNRQKIRSLDVVGKIRREIQNLKLFRHPHIIKLYQVISTPSDIFMVMEYVSGGELFDYICKNGRLDEKESRRLFQQILSGVDYCHRHMVVHRDLKPENVLLDAHMNAKIADFGLSNMMSDGEFLRTSCGSPNYAAPEVISGRLYAGPEVDIWSSGVILYALLCGTLPFDDDHVPTLFKKICDGIFYTPQYLNPSVISLLKHMLQVDPMKRATIKDIREHEWFKQDLPKYLFPEDPSYSSTMIDDEALKEVCEKFECSEEEVLSCLYNRNHQDPLAVAYHLIIDNRRIMNEAKDFYLATSPPDSFLDDHHLTRPHPERVPFLVAETPRARHTLDELNPQKSKHQGVRKAKWHLGIRSQSRPNDIMAEVCRAIKQLDYEWKVVNPYYLRVRRKNPVTSTYSKMSLQLYQVDSRTYLLDFRSIDDEITEAKSGTATPQRSGSVSNYRSCQRSDSDAEAQGKSSEVSLTSSVTSLDSSPVDLTPRPGSHTIEFFEMCANLIKILAQ

SEQ ID NO: 42 - protein, 552 aa, Homo sapiens
MAEKQKHDGRVKIGHYVLGDTLGVGTFGKVKIGEHQLTGHKVAVKILNRQKIRSLDVVGKIKREIQNLKLFRHPHIIKLYQVISTPTDFFMVMEYVSGGELFDYICKHGRVEEMEARRLFQQILSAVDYCHRHMVVHRDLKPENVLLDAHMNAKIADFGLSNMMSDGEFLRTSCGSPNYAAPEVISGRLYAGPEVDIWSCGVILYALLCGTLPFDDEHVPTLFKKIRGGVFYIPEYLNRSVATLLMHMLQVDPLKRATIKDIREHEWFKQDLPSYLFPEDPSYDANVIDDEAVKEVCEKFECTESEVMNSLYSGDPQDQLAVAYHLIIDNRRIMNQASEFYLASSPPSGSFMDDSAMHIPPGLKPHPERMPPLIADSPKARCPLDALNTTKPKSLAVKKAKWHLGIRSQSKPYDIMAEVYRAMKQLDFEWKVVNAYHLRVRRKNPVTGNYVKMSLQLYLVDNRSYLLDFKSIDDEVVEQRSGSSTPQRSCSAAGLHRPRSSFDSTTAESHSLSGSLTGSLTGSTLSSVSPRLGSHTMDFFEMCASLITTLAR